SEQUENCE LISTING (16 sequences)

SEQ ID NO: 1 - DNA, 20 bp, Artificial Sequence
  Synthetically generated oligonucleotide
tccatgacgttcctgacgtt

SEQ ID NO: 2 - DNA, 18 bp, Artificial Sequence
  Synthetically generated oligonucleotide
tctcccagcgtgcgccat

SEQ ID NO: 3 - DNA, 30 bp, Artificial Sequence
  Synthetically generated oligonucleotide
accgatgacgtcgccggtgacggcaccacg

SEQ ID NO: 4 - DNA, 24 bp, Artificial Sequence
  Synthetically generated oligonucleotide
tcgtcgttttgtcgttttgtcgtt

SEQ ID NO: 5 - DNA, 20 bp, Artificial Sequence
  Synthetically generated oligonucleotide
tccatgacgttcctgatgct

SEQ ID NO: 6 - DNA, 22 bp, Artificial Sequence
  Synthetically generated oligonucleotide
tcgacgttttcggcgcgcgccg

SEQ ID NO: 7 - DNA, 3411 bp, Artificial Sequence
  F4co nucleotide
atggtcattgttcagaacatacagggccaaatggtccaccaggcaattagtccgcgaactcttaatgcatgggtgaaggtcgtggaggaaaaggcattctccccggaggtcattccgatgttttctgcgctatctgagggcgcaacgccgcaagaccttaataccatgcttaacacggtaggcgggcaccaagccgctatgcaaatgctaaaagagactataaacgaagaggccgccgaatgggatcgagtgcacccggtgcacgccggcccaattgcaccaggccagatgcgcgagccgcgcgggtctgatattgcaggaactacgtctacccttcaggagcagattgggtggatgactaacaatccaccaatcccggtcggagagatctataagaggtggatcatactgggactaaacaagatagtccgcatgtattctccgacttctatactggatatacgccaaggcccaaaggagccgttcagggactatgtcgaccgattctataagacccttcgcgcagagcaggcatcccaggaggtcaaaaattggatgacagaaactcttttggtgcagaatgcgaatccggattgtaaaacaattttaaaggctctaggaccggccgcaacgctagaagagatgatgacggcttgtcagggagtcggtggaccggggcataaagcccgcgtcttacacatgggcccgatatctccgatagaaacagtttcggtcaagcttaaaccagggatggatggtccaaaggtcaagcagtggccgctaacggaagagaagattaaggcgctcgtagagatttgtactgaaatggagaaggaaggcaagataagcaagatcgggccagagaacccgtacaatacaccggtatttgcaataaagaaaaaggattcaacaaaatggcgaaagcttgtagattttagggaactaaacaagcgaacccaagacttttgggaagtccaactagggatcccacatccagccggtctaaagaagaagaaatcggtcacagtcctggatgtaggagacgcatattttagtgtaccgcttgatgaggacttccgaaagtatactgcgtttactataccgagcataaacaatgaaacgccaggcattcgctatcagtacaacgtgctcccgcagggctggaaggggtctccggcgatatttcagagctgtatgacaaaaatacttgaaccattccgaaagcagaatccggatattgtaatttaccaatacatggacgatctctatgtgggctcggatctagaaattgggcagcatcgcactaagattgaggaactgaggcaacatctgcttcgatggggcctcactactcccgacaagaagcaccagaaggagccgccgttcctaaagatgggctacgagcttcatccggacaagtggacagtacagccgatagtgctgcccgaaaaggattcttggaccgtaaatgatattcagaaactagtcggcaagcttaactgggcctctcagatttacccaggcattaaggtccgacagctttgcaagctactgaggggaactaaggctctaacagaggtcatcccattaacggaggaagcagagcttgagctggcagagaatcgcgaaattcttaaggagccggtgcacggggtatactacgacccctccaaggaccttatagccgagatccagaagcaggggcagggccaatggacgtaccagatatatcaagaaccgtttaagaatctgaagactgggaagtacgcgcgcatgcgaggggctcatactaatgatgtaaagcaacttacggaagcagtacaaaagattactactgagtctattgtgatatggggcaagaccccaaagttcaagctgcccatacagaaggaaacatgggaaacatggtggactgaatattggcaagctacctggattccagaatgggaatttgtcaacacgccgccacttgttaagctttggtaccagcttgaaaaggagccgatagtaggggcagagaccttctatgtcgatggcgccgcgaatcgcgaaacgaagctaggcaaggcgggatacgtgactaataggggccgccaaaaggtcgtaacccttacggataccaccaatcagaagactgaactacaagcgatttaccttgcacttcaggatagtggcctagaggtcaacatagtcacggactctcaatatgcgcttggcattattcaagcgcagccagatcaaagcgaaagcgagcttgtaaaccaaataatagaacagcttataaagaaagagaaggtatatctggcctgggtccccgctcacaagggaattggcggcaatgagcaagtggacaagctagtcagcgctgggattcgcaaggttcttgcgatggggggtaagtggtctaagtctagcgtagtcggctggccgacagtccgcgagcgcatgcgacgcgccgaaccagccgcagatggcgtgggggcagcgtctagggatctggagaagcacggggctataacttccagtaacacggcggcgacgaacgccgcatgcgcatggttagaagcccaagaagaggaagaagtagggtttccggtaactccccaggtgccgttaaggccgatgacctataaggcagcggtggatctttctcacttccttaaggagaaaggggggctggagggcttaattcacagccagaggcgacaggatattcttgatctgtggatttaccatacccaggggtactttccggactggcagaattacaccccggggccaggcgtgcgctatcccctgactttcgggtggtgctacaaactagtcccagtggaacccgacaaggtcgaagaggctaataagggcgagaacacttctcttcttcacccggtaagcctgcacgggatggatgacccagaacgagaggttctagaatggaggttcgactctcgacttgcgttccatcacgtagcacgcgagctgcatccagaatatttcaagaactgccgcccaatgggcgccagggccagtgtacttagtggcggagaactagatcgatgggaaaagatacgcctacgcccggggggcaagaagaagtacaagcttaagcacattgtgtgggcctctcgcgaacttgagcgattcgcagtgaatccaggcctgcttgagacgagtgaaggctgtaggcaaattctggggcagctacagccgagcctacagactggcagcgaggagcttcgtagtctttataataccgtcgcgactctctactgcgttcatcaacgaattgaaataaaggatactaaagaggcccttgataaaattgaggaggaacagaataagtcgaaaaagaaggcccagcaggccgccgccgacaccgggcacagcaaccaggtgtcccaaaactactaa

SEQ ID NO: 8 - protein, 1136 aa, Artificial Sequence
  F4co protein
MVIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLHMGPISPIETVSVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLAMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCRPMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNY

SEQ ID NO: 9 - DNA, 1302 bp, Artificial Sequence
  p51 RT nucleotide
atgagtactggtccgatctctccgatagaaacagtttcggtcaagcttaaaccagggatggatggtccaaaggtcaagcagtggccgctaacggaagagaagattaaggcgctcgtagagatttgtactgaaatggagaaggaaggcaagataagcaagatcgggccagagaacccgtacaatacaccggtatttgcaataaagaagaaggattcaacaaaatggcgaaagcttgtagattttagggaactaaacaagcgaacccaagacttttgggaagtccaactaggtatcccacatccagccggtctaaagaagaagaaatcggtcacagtcctggatgtaggagacgcatattttagtgtaccgcttgatgaggacttccgaaagtatactgcgtttactataccgagcataaacaatgaaacgccaggcattcgctatcagtacaacgtgctcccgcagggctggaaggggtctccggcgatatttcagagctctatgacaaaaatacttgaaccattccgaaagcagaatccggatattgtaatttaccaatacatggacgatctctatgtgggctcggatctagaaattgggcagcatcgcactaagattgaggaactgaggcaacatctgcttcgatggggcctcactactcccgacaagaagcaccagaaggagccgccgttcctaaagatgggctacgagcttcatccggacaagtggacagtacagccgatagtgctgcccgaaaaggattcttggaccgtaaatgatattcagaaactagtcggcaagcttaactgggcctctcagatttacccaggcattaaggtccgacagctttgcaagctactgaggggaactaaggctctaacagaggtcatcccattaacggaggaagcagagcttgagctggcagagaatcgcgaaattcttaaggagccggtgcacggggtatactacgacccctccaaggaccttatagccgagatccagaagcaggggcagggccaatggacgtaccagatatatcaagaaccgtttaagaatctgaagactgggaagtacgcgcgcatgcgaggggctcatactaatgatgtaaagcaacttacggaagcagtacaaaagattactactgagtctattgtgatatggggcaagaccccaaagttcaagctgcccatacagaaggaaacatgggaaacatggtggactgaatattggcaagctacctggattccagaatgggaatttgtcaacacgccgccgctggtaaaactgaggcctgctagctaa

SEQ ID NO: 10 - protein, 433 aa, Artificial Sequence
  p51 RT protein
MSTGPISPIETVSVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLRPAS

SEQ ID NO: 11 - DNA, 3350 bp, Artificial Sequence
  F4fs nucleotide
tggtcattgttcagaacatacagggccaaatggtccaccaggcaattagtccgcgaactcttaatgcatgggtgaaggtcgtggaggaaaaggcattctccccggaggtcattccgatgttttctgcgctatctgagggcgcaacgccgcaagaccttaataccatgcttaacacggtaggcgggcaccaagccgctatgcaaatgctaaaagagactataaacgaagaggccgccgaatgggatcgagtgcacccggtgcacgccggcccaattgcaccaggccagatgcgcgagccgcgcgggtctgatattgcaggaactacgtctacccttcaggagcagattgggtggatgactaacaatccaccaatcccggtcggagagatctataagaggtggatcatactgggactaaacaagatagtccgcatgtattctccgacttctatactggatatacgccaaggcccaaaggagccgttcagggactatgtcgaccgattctataagacccttcgcgcagagcaggcatcccaggaggtcaaaaattggatgacagaaactcttttggtgcagaatgcgaatccggattgtaaaacaattttaaaggctctaggaccggccgcaacgctagaagagatgatgacggcttgtcagggagtcggtggaccggggcataaagcccgcgtcttacacatgggcccgatatctccgatagaaacagtttcggtcaagcttaaaccagggatggatggtccaaaggtcaagcagtggccgctaacggaagagaagattaaggcgctcgtagagatttgtactgaaatggagaaggaaggcaagataagcaagatcgggccagagaacccgtacaatacaccggtatttgcaataaagaaaaaggattcaacaaaatggcgaaagcttgtagattttagggaactaaacaagcgaacccaagacttttgggaagtccaactagggatcccacatccagccggtctaaagaagaagaaatcggtcacagtcctggatgtaggagacgcatattttagtgtaccgcttgatgaggacttccgaaagtatactgcgtttactataccgagcataaacaatgaaacgccaggcattcgctatcagtacaacgtgctcccgcagggctggaaggggtctccggcgatatttcagagctgtatgacaaaaatacttgaaccattccgaaagcagaatccggatattgtaatttaccaatacatggacgatctctatgtgggctcggatctagaaattgggcagcatcgcactaagattgaggaactgaggcaacatctgcttcgatggggcctcactactcccgacaagaagcaccagaaggagccgccgttcctaaagatgggctacgagcttcatccggacaagtggacagtacagccgatagtgctgcccgaaaaggattcttggaccgtaaatgatattcagaaactagtcggcaagcttaactgggcctctcagatttacccaggcattaaggtccgacagctttgcaagctactgaggggaactaaggctctaacagaggtcatcccattaacggaggaagcagagcttgagctggcagagaatcgcgaaattcttaaggagccggtgcacggggtatactacgacccctccaaggaccttatagccgagatccagaagcaggggcagggccaatggacgtaccagatatatcaagaaccgtttaagaatctgaagactgggaagtacgcgcgcatgcgaggggctcatactaatgatgtaaagcaacttacggaagcagtacaaaagattactactgagtctattgtgatatggggcaagaccccaaagttcaagctgcccatacagaaggaaacatgggaaacatggtggactgaatattggcaagctacctggattccagaatgggaatttgtcaacacgccgccacttgttaagctttggtaccagcttgaaaaggagccgatagtaggggcagagaccttctatgtcgatggcgccgcgaatcgcgaaacgaagctaggcaaggcgggatacgtgactaataggggccgccaaaaggtcgtaacccttacggataccaccaatcagaagactgaactacaagcgatttaccttgcacttcaggatagtggcctagaggtcaacatagtcacggactctcaatatgcgcttggcattattcaagcgcagccagatcaaagcgaaagcgagcttgtaaaccaaataatagaacagcttataaagaaagagaaggtatatctggcctgggtccccgctcacaagggaattggcggcaatgagcaagtggacaagctagtcagcgctgggattcgcaaggttcttgccatggggggtaagtggtctaagtctagcgtagtcggctggccgacagtccgcgagcgcatgcgacgcgccgaaccagccgcagatggcgtgggggcagcgtctagggatctggagaagcacggggctataacttccagtaacacggcggcgacgaacgccgcatgcgcatggttagaagcccaagaagaggaagaagtagggtttccggtaactccccaggtgccgttaaggccgatgacctataaggcagcggtggatctttctcacttccttaaggagaaaggggggctggagggcttaattcacagccagaggcgacaggatattcttgatctgtggatttaccatacccaggggtactttccggactggcagaattacaccccggggccaggcgtgcgctatcccctgactttcgggtggtgctacaaactagtcccagtggaacccgacaaggtcgaagaggctaataagggcgagaacacttctcttcttcacccggtaagcctgcacgggatggatgacccagaacgagaggttctagaatggaggttcgactctcgacttgcgttccatcacgtagcacgcgagctgcatccagaatatttcaagaactgccgcccaatgggcgccagggccagtgtacttagtggcggagaactagatcgatgggaaaagatacgcctacgcccggggggcaagaagaagtacaagcttaagcacattgtgtgggcctctcgcgaacttgagcgattcgcagtgaatccaggcctgcttgagacgagtgaaggctgtaggcaaattctggggcagctacagccgagcctacagactggcagcgaggagcttcgtagtctttataataccgtcgcgactctctactgcgttcatcaacgaattgaaataaaggatactaaagaggcccttgataaaattgaggaggaacagaataagtcgtaatga

SEQ ID NO: 12 - protein, 1115 aa, Artificial Sequence
  F4fs protein
MVIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLHMGPISPIETVSVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSCMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLAMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCRPMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKS

SEQ ID NO: 13 - DNA, 3423 bp, Artificial Sequence
  C F4.His nucleotide
atggttattgttcagaatctgcagggtcagatggttcatcaggcaatttctccgcgtaccctgaatgcatgggtgaaagtgattgaagaaaaagccttttctccggaagttattccgatgtttaccgcactgagcgaaggtgcaacaccgcaggatctgaataccatgctgaataccgttggtggtcatcaggcagcaatgcagatgctgaaagataccattaatgaagaggcagcagaatgggatcgtctgcatccggttcatgcaggtccgattgcaccgggtcagatgcgtgaaccgcgtggtagcgatattgcaggtacaaccagcaccctgcaagagcagattgcatggatgaccagcaatcctccgattccggttggtgatatttataaacgctggattattctgggcctgaataaaattgtgcgtatgtattctccggttagcattctggatattaaacagggtccgaaagaaccgtttcgtgattatgtggatcgcttttttaaaaccctgcgtgcagaacaggcaacccaagaggttaaaaattggatgaccgataccctgctggttcagaatgcaaatccggattgcaaaaccattctgcgtgcactgggtccgggtgcaacactggaagaaatgatgaccgcatgtcagggtgttggtggtccgggtcataaagcacgtgttctgcacatgggtccgattagcccgattgaaaccgttccggtgaaactgaaaccgggtatggatggtccgaaagttaaacagtggcctctgaccgaagaaaaaatcaaagccctgaccgcaatttgtgaagaaatggaaaaagaaggcaaaattaccaaaattggtccggaaaatccgtataacacaccggtgtttgccattaaaaaaaaagatagcaccaaatggcgtaaactggtggattttcgcgaactgaataaacgtacccaggatttttgggaagttcagctgggtattccgcatccggcaggtctgaaaaaaaaaaaatccgtgaccgttctggatgttggtgatgcctatttttctgttccgctggatgaaggttttcgtaaatataccgcctttaccattccgagcattaataatgaaacaccgggtattcgctatcagtataatgttctgccgcagggttggaaaggttctccggcaatttttcagagcagcatgaccaaaattctggaaccgtttcgcgcacagaatccggaaattgtgatttatcagtatatggatgatctgtatgttggtagcgatctggaaattggtcagcatcgtgccaaaattgaagaactgcgtgaacatctgctgaaatggggttttaccacaccggataaaaaacatcagaaagaaccgccgtttctgaaaatgggttatgaactgcatccggataaatggaccgttcagccgattcagctgccggaaaaagatagctggaccgtgaatgatattcagaaactggtgggcaaactgaattgggcaagccagatttatccgggtattaaagttcgtcagctgtgtaaactgctgcgtggtgcaaaagcactgaccgatattgttccgctgacagaagaagcagaactggaactggccgaaaatcgtgaaattctgaaagaaccggtgcatggtgtttattatgatccgagcaaagatctgattgccgaaattcagaaacagggtcatgatcagtggacctatcagatttatcaggaaccgtttaaaaatctgaaaaccggcaaatatgcaaaaatgcgtaccgcacataccaatgatgttaaacagctgaccgaagccgttcagaaaattgccatggaaagcattgtgatttggggtaaaacaccgaaatttcgtctgccgattcagaaagaaacctgggaaacatggtggaccgattattggcaggcaacctggattccggaatgggaatttgttaatacaccgccgctggttaaactgtggtatcagctggaaaaagaaccgattgcaggtgcagaaaccttttatgttgatggtgcagcaaatcgcgaaaccaaaattggcaaagccggttatgttaccgatcgtggtcgtcagaaagttgttagcctgaccgaaaccaccaatcagaaaaccgaactgcaggcaattcagctggccctgcaggatagcggtagcgaagttaatattgtgaccgatagccagtatgcactgggtattattcaggcacagccggataaaagcgaaagcgaactggtgaatcagattattgaacagctgattaaaaaagaacgcgtgtatctgagctgggttccggcacataaaggtattggtggcaatgaacaggttgataaactggttagcagcggtattcgtaaagttctggccatgggtggtaaatggtctaaaagcagcattgttggttggccggcaattcgtgaacgtatgcgtcgtaccgaaccggcagcagaaggtgttggcgcagcaagccaggatctggataaacatggtgcactgaccagcagcaataccgcaaccaataatgcagattgtgcatggctggaagcacaggaagaagaagaagaagttggttttccggttcgtccgcaggttccgctgcgtccgatgacctataaagcagcatttgatctgagcttttttctgaaagaaaaaggtggtctggaaggtctgatttatagcaaaaaacgccaggatattctggatctgtgggtttatcatacccagggtttttttccggattggcagaattacacaccgggtccgggtgtgcgttatccgctgacctttggttggtgttataaactggttccggttgatccgcgtgaagttgaagaagcaaacgaaggcgaaaataattgtctgctgcatccgatgagccagcatggtatggaagatgaagatcgcgaagtgctgaaatggaaatttgatagccatctggctcgtcgtcacatggcacgcgaactgcatccggaatattataaagattgccgtccgatgggtgcacgtgcaagcattctgcgtggtggtaaactggataaatgggaaaaaattcgtctgcgtccgggtggtaaaaaacattatatgctgaaacatctggtttgggcaagccgtgaactggaacgttttgcactgaatccgggtctgctggaaaccagcgaaggttgcaaacaaattattaaacagctgcagccggcactgcagaccggcaccgaagaactgcgcagcctgtataataccgttgcaaccctgtattgtgtgcatgcgaaaattgaagtgcgcgataccaaagaagcactggataaaattgaagaagaacagaataaaagccagcagaaaacccagcaggcaaaagcagcagatggtaaagtgagccagaattatcaccaccaccaccaccactaa

SEQ ID NO: 14 - protein, 1140 aa, Artificial Sequence
  C F4.His protein
MVIVQNLQGQMVHQAISPRTLNAWVKVIEEKAFSPEVIPMFTALSEGATPQDLNTMLNTVGGHQAAMQMLKDTINEEAAEWDRLHPVHAGPIAPGQMREPRGSDIAGTTSTLQEQIAWMTSNPPIPVGDIYKRWIILGLNKIVRMYSPVSILDIKQGPKEPFRDYVDRFFKTLRAEQATQEVKNWMTDTLLVQNANPDCKTILRALGPGATLEEMMTACQGVGGPGHKARVLHMGPISPIETVPVKLKPGMDGPKVKQWPLTEEKIKALTAICEEMEKEGKITKIGPENPYNTPVFAIKKKDSTKWRKLVDFRELNKRTQDFWEVQLGIPHPAGLKKKKSVTVLDVGDAYFSVPLDEGFRKYTAFTIPSINNETPGIRYQYNVLPQGWKGSPAIFQSSMTKILEPFRAQNPEIVIYQYMDDLYVGSDLEIGQHRAKIEELREHLLKWGFTTPDKKHQKEPPFLKMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGHDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKEPIAGAETFYVDGAANRETKIGKAGYVTDRGRQKVVSLTETTNQKTELQAIQLALQDSGSEVNIVTDSQYALGIIQAQPDKSESELVNQIIEQLIKKERVYLSWVPAHKGIGGNEQVDKLVSSGIRKVLAMGGKWSKSSIVGWPAIRERMRRTEPAAEGVGAASQDLDKHGALTSSNTATNNADCAWLEAQEEEEEVGFPVRPQVPLRPMTYKAAFDLSFFLKEKGGLEGLIYSKKRQDILDLWVYHTQGFFPDWQNYTPGPGVRYPLTFGWCYKLVPVDPREVEEANEGENNCLLHPMSQHGMEDEDREVLKWKFDSHLARRHMARELHPEYYKDCRPMGARASILRGGKLDKWEKIRLRPGGKKHYMLKHLVWASRELERFALNPGLLETSEGCKQIIKQLQPALQTGTEELRSLYNTVATLYCVHAKIEVRDTKEALDKIEEEQNKSQQKTQQAKAADGKVSQNYHHHHHH

SEQ ID NO: 15 - DNA, 3405 bp, Artificial Sequence
  C F4 nucleotide
atggtcatcgtgcagaatctgcagggccagatggtgcaccaggccatctctcccagaaccctgaacgcctgggtgaaagtgatcgaggaaaaagccttcagccccgaagtgatccccatgttcaccgccctgagcgaaggcgccaccccccaggacctgaacaccatgctgaacaccgtgggaggacaccaggccgccatgcagatgctgaaggacaccatcaacgaagaggccgccgagtgggacagactgcaccctgtgcacgccggacctatcgcccctggccagatgagagagcccagaggcagcgatatcgccggcaccacaagcaccctgcaggaacagatcgcctggatgaccagcaacccccccatccccgtgggagacatctacaagcggtggatcatcctgggcctgaacaagatcgtgcggatgtacagccccgtgtccatcctggacatcaagcagggccccaaagagcccttccgggactacgtggaccggttcttcaagaccctgcgggccgagcaggccacccaggaagtgaagaactggatgaccgacaccctgctggtgcagaacgccaaccccgactgcaagaccatcctgagagccctgggacctggcgccaccctggaagagatgatgaccgcctgtcagggcgtcggcggacctggacacaaggcccgggtgctgcacatgggacccatctcccccatcgagacagtgcccgtgaagctgaagcccggcatggacggccccaaagtgaagcagtggcccctgaccgaggaaaagatcaaggccctgaccgccatctgcgaggaaatggaaaaagagggcaagatcaccaagatcggccccgagaatccctacaacacccccgtgttcgccatcaagaagaaagacagcaccaagtggcggaaactggtggacttccgggagctgaacaagcggacccaggacttctgggaggtgcagctgggcatccctcaccctgccggcctgaagaaaaagaaaagcgtgaccgtgctggacgtgggcgacgcctacttcagcgtgcccctggacgagggcttcagaaagtacaccgccttcaccatccccagcatcaacaacgagacacccggcatcagataccagtacaacgtgctgccccagggctggaagggcagccccgccatcttccagagcagcatgaccaagatcctggaacccttccgggcccagaaccccgagatcgtgatctaccagtacatggacgacctgtacgtgggcagcgacctggaaatcggccagcaccgggccaagatcgaggaactgcgggagcatctgctgaagtggggcttcaccacccccgacaagaagcaccagaaagagccccccttcctgaagatgggctacgagctgcaccccgacaagtggaccgtgcagcccatccagctgcccgagaaggacagctggaccgtgaacgacatccagaaactcgtgggcaagctgaactgggccagccagatctaccccggcatcaaagtgcggcagctgtgcaagctcctgagaggcgccaaagccctgaccgatatcgtgcctctgacagaggaagccgagctggaactggccgagaacagagagatcctgaaagaacccgtgcatggcgtgtactacgaccccagcaaggacctgatcgccgagatccagaagcagggccacgaccagtggacctaccagatctatcaggaacccttcaagaacctcaagaccggcaagtacgccaagatgcggaccgcccacaccaacgacgtgaagcagctgaccgaggccgtgcagaaaatcgccatggaaagcatcgtgatctggggcaagacacccaagttccggctgcccatccagaaagagacatgggagacttggtggaccgactactggcaggccacctggatccccgagtgggagttcgtgaacaccccccctctggtcaagctgtggtatcagctcgagaaagagcctatcgctggcgccgagacattctacgtggacggcgctgccaaccgggagacaaagatcggcaaggccggctacgtgaccgaccggggcagacagaaagtggtgtccctgaccgaaaccaccaaccagaaaaccgagctgcaggccattcagctggcactgcaggatagcggcagcgaagtgaacatcgtgaccgacagccagtacgccctgggcatcatccaggcccagcccgacaagagcgagagcgagctggtcaaccagatcatcgagcagctgatcaagaaagaacgggtgtacctgagctgggtgcccgcccacaagggcatcggcggaaacgaacaggtggacaagctggtgtccagcggcatccggaaggtcctggccatgggtggcaagtggagcaagagcagcatcgtgggctggcccgccatccgggagcggatgagaagaaccgagcctgccgccgaaggcgtgggagctgccagccaggatctggataagcacggcgccctgaccagcagcaacaccgccaccaacaacgccgactgcgcctggctggaagcccaggaagaggaagaagaagtcggcttcccagtcagacctcaggtgcccctgcggcccatgacctacaaggccgccttcgacctgagcttcttcctgaaagagaagggcggcctggaaggcctgatctacagcaagaagcggcaggacatcctggacctgtgggtgtaccacacccagggcttctttccagactggcagaactacacccccggacccggcgtgagataccccctgaccttcggctggtgctacaaactggtgcccgtggaccccagagaggtggaggaagccaacgagggcgagaacaactgcctgctgcaccccatgagccagcacggcatggaagatgaggaccgggaggtgctgaaatggaagttcgacagccacctggccagacggcacatggccagagagctgcatcccgagtactacaaggactgccggcctatgggcgccagagcctctatcctgagaggcggcaagctggataagtgggagaagatccggctgcggcctggcggcaagaaacactacatgctgaagcacctcgtgtgggccagccgggagctggaaagattcgccctgaaccccggactgctggaaaccagcgagggctgcaagcagatcatcaaacagctgcagcccgccctgcagacaggcaccgaggaactgagaagcctgtacaacaccgtggccaccctgtactgcgtgcatgccaagattgaagtgcgggacaccaaagaggccctggacaagatcgaagaggaacagaacaagagccagcagaaaacccagcaggccaaggccgccgatggcaaggtgtcccagaactactga

SEQ ID NO: 16 - protein, 1134 aa, Artificial Sequence
  C F4 protein
MVIVQNLQGQMVHQAISPRTLNAWVKVIEEKAFSPEVIPMFTALSEGATPQDLNTMLNTVGGHQAAMQMLKDTINEEAAEWDRLHPVHAGPIAPGQMREPRGSDIAGTTSTLQEQIAWMTSNPPIPVGDIYKRWIILGLNKIVRMYSPVSILDIKQGPKEPFRDYVDRFFKTLRAEQATQEVKNWMTDTLLVQNANPDCKTILRALGPGATLEEMMTACQGVGGPGHKARVLHMGPISPIETVPVKLKPGMDGPKVKQWPLTEEKIKALTAICEEMEKEGKITKIGPENPYNTPVFAIKKKDSTKWRKLVDFRELNKRTQDFWEVQLGIPHPAGLKKKKSVTVLDVGDAYFSVPLDEGFRKYTAFTIPSINNETPGIRYQYNVLPQGWKGSPAIFQSSMTKILEPFRAQNPEIVIYQYMDDLYVGSDLEIGQHRAKIEELREHLLKWGFTTPDKKHQKEPPFLKMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGHDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKEPIAGAETFYVDGAANRETKIGKAGYVTDRGRQKVVSLTETTNQKTELQAIQLALQDSGSEVNIVTDSQYALGIIQAQPDKSESELVNQIIEQLIKKERVYLSWVPAHKGIGGNEQVDKLVSSGIRKVLAMGGKWSKSSIVGWPAIRERMRRTEPAAEGVGAASQDLDKHGALTSSNTATNNADCAWLEAQEEEEEVGFPVRPQVPLRPMTYKAAFDLSFFLKEKGGLEGLIYSKKRQDILDLWVYHTQGFFPDWQNYTPGPGVRYPLTFGWCYKLVPVDPREVEEANEGENNCLLHPMSQHGMEDEDREVLKWKFDSHLARRHMARELHPEYYKDCRPMGARASILRGGKLDKWEKIRLRPGGKKHYMLKHLVWASRELERFALNPGLLETSEGCKQIIKQLQPALQTGTEELRSLYNTVATLYCVHAKIEVRDTKEALDKIEEEQNKSQQKTQQAKAADGKVSQNY

(19) United States
(12) Patent Application Publication
(10) Pub. No.: US 2013/0280322 A1
(43) Pub. Date: Oct. 24, 2013

(54) VACCINE

(71) Applicants: Marguerite Christine Koutsoukos, Research Triangle Park, NC (US); Patricia B. Bourguignon, Research Triangle Park, NC (US); Gerald Hermann Voss, Research Triangle Park, NC (US)

(72) Inventors: Marguerite Christine Koutsoukos, Research Triangle Park, NC (US); Patricia B. Bourguignon, Research Triangle Park, NC (US); Gerald Hermann Voss, Research Triangle Park, NC (US)

(73) Assignee: GlaxoSmithKline Biologicals S.A., Rixensart (BE)

(21) Appl. No.: 13/825,983

(22) PCT Filed: Sep. 27, 2011
(86) PCT No.: PCT/EP2011/066754

§ 371 Date: Mar. 26, 2013

Related U.S. Application Data
(60) Provisional application No. 61/386,931, filed on Sep. 27, 2010.

(51) Int. Cl. A61K 39/21

(52) U.S. Cl. CPC A61K 39/21
USPC 424/450; 424/188.1

(57) ABSTRACT

The present invention provides methods and compositions for the treatment of HIV-1 infected subjects. The invention relates in particular to enhancing the immune response of an infected subject and to stabilising or reducing the viral load of an infected subject.

### FIELD OF THE INVENTION

The present invention relates to immunogenic compositions comprising HIV-1 antigens and uses thereof in the treatment of HIV-1 infected subjects. In particular, the invention relates to the use of immunogenic compositions comprising HIV-1 antigens to treat or ameliorate disease in HIV-1 infected subjects and/or to delay or mitigate the need for anti-retroviral therapy by HIV-1 infected subjects.

### BACKGROUND TO THE INVENTION

There are a number of immunological features of HIV pathogenicity that makes HIV vaccine development a challenging task. There is a high probability that antibodies and T-cells select successive immunological variants of the virus which continue to evade the host response. There is evidence for the existence of specific immunity following natural infection, and the virus is held in check by the host immune response, often for many years, until the immune system is finally incapable of containing the virus. During the course of infection with HIV, cellular immune responses (helper and specific cytotoxic T-cells) as well as binding antibodies, virus neutralization, and antibody-dependent cell mediated cytotoxicity (ADCC) can be detected [Letvin, 1993; Weiss, 1993].

The typical course of HIV-1 infection in untreated individuals follows three major phases, generally over a period of 8 to 12 years (FIG. 1) [Pantaleo, 1993]. Although highly variable between individuals, the pattern and course of the infection is as follows:

Acute (primary) infection: Primary HIV-1 infection is a transient condition. It is a symptomatic illness in 40-90% of patients, accompanied by i) an initial rapid rise in plasma viremia, ii) a decrease in blood CD4+ T-lymphocyte count and iii) a large increase in blood CD8+ T-lymphocyte count. The decline of the initial rise in plasma viremia generally correlates with the appearance of virus-specific immune responses particularly HIV-1-specific cytotoxic T-lymphocytes (CTLs) [Kaufmann, 1999].

Chronic, asymptomatic phase: During this period, clinical but not virological latency occurs (median time 10 years) with a relatively stable level of CD4+ T-lymphocyte count and low viral loads. This phase is an important target for disease management, as the subject typically remains healthy. Long-term nonprogressors (LTNP) can remain in this phase without treatment for years. Current disease management strategies, such as ART, prolong this phase for as long as possible.

Chronic, symptomatic HIV infection (AIDS): Clinical AIDS begins with the onset of the first CDC Class C [CDC, 1993] or WHO stage IV clinical event [WHO, 2005]. AIDS signals the end stage of HIV-1 infection with death occurring within 2 to 3 years in the absence of anti-retroviral therapy.

Therapeutic vaccines administered to HIV-1 infected subjects are useful for stabilising or reducing HIV-1 viral load and therefore slow progression of HIV disease while reducing or eliminating the need for additional antiviral treatments. Therefore, there remains a need for a therapeutic vaccine to manage HIV disease in infected subjects.

### SUMMARY OF THE INVENTION

It is an object of the invention to provide methods and compositions to enhance the T cell response of an HIV-1 infected subject comprising

1) selecting a subject infected with HIV-1; and

2) administering to the subject an immunogenic composition comprising
a. two or more HIV-1 antigens selected from the group consisting of Nef, Gag, and Pol;
b. an adjuvant that induces a Th1 immune response; and
c. a pharmaceutically acceptable excipient.

It is a further object of the invention to provide methods and compositions for inducing an immune response in a subject infected with HIV-1, wherein after administration of the composition, a higher percentage of CD4+ T cells from the subject show specific recognition of at least one polypeptide of the immunogenic composition is increased as compared to before the administration.

It is a further object of the invention to provide methods and compositions for inducing an immune response in a subject infected with HIV-1, wherein after administration of the composition, a higher percentage of CD4+ T cells from the subject express at least one, two or three activation markers selected from the group consisting of CD40L, IL-2, TNFα and IFNγ as compared to before administration.

It is a further object of the invention to provide methods and compositions for restoring or inhibiting the loss of HIV-1 specific CD8+ T cell function in a subject infected with HIV-1 comprising administration of the composition of the present invention.

It is a further object of the invention to provide methods and compositions for reducing the increase in the viral load in a subject infected with HIV-1, wherein after administration of the composition, the viral load of the subject remains stable or decreases for at least four months as compared to before administration.

It is a further object of the invention to provide methods and compositions for the prevention of the onset of clinical HIV disease in a subject infected with HIV-1, wherein after administration of the composition, the viral load of the subject remains below 100,000 copies/ml for at least four months after administration.

Also provided is a pharmaceutical composition comprising
a. two or more HIV-1 antigens selected from the group consisting of Nef, Gag, and Pol;
b. an adjuvant that induces a Th1 immune response; and
c. a pharmaceutically acceptable excipient,
   for use in the reduction or maintenance of the viral load of an HIV-1 infected subject at or below 100,000 copies/ml for at least four months after administration in the absence of antiretroviral therapy (ART). In one embodiment, the subject is not on ART.

In one embodiment, the pharmaceutical composition is for maintaining the stability of the viral load of an HIV-1 infected subject in the absence of ART for at least four months.

In another embodiment, the pharmaceutical composition is for use in enhancing the T cell response of an HIV-1 infected subject not on ART. In a further embodiment, the enhanced T cell response is a higher percentage of either CD4+ T cells or CD8+ T cells from the subject that show specific recognition of at least one polypeptide of the pharmaceutical composition as compared to before administration of the pharmaceutical composition. In a further embodiment, the enhanced T cell response is a higher percentage of CD4+ T cells from the subject that express at least one, two or three activation markers, such as CD40L, IL-2, TNFα and IFNγ, as compared to before administration of the pharmaceutical composition. In a further embodiment, the enhanced T cell response is the restoration or inhibition of loss of HIV-1 specific CD8+ T cell function.

In a further embodiment, the viral load of the subject remains below 100,000 copies/ml for at least six months, at least twelve months, at least eighteen months, at least two years, at least three years, at least four years, at least five years, at least six years, at least seven years, at least eight years, at least nine years, or at least ten years. In another embodiment, the subject maintains a viral load below 50,000 copies/ml, below 10,000 copies/ml, below 5000 copies/ml, below 1000 copies/ml, or below 500 copies/ml.

In one embodiment, the subject is concurrently treated with anti-retroviral therapy (ART). In a further embodiment, the subject discontinues anti-retroviral therapy (ART) prior to or subsequent to administration of said immunogenic composition. In another embodiment, the subject is ART-naïve. In a further embodiment, the subject abstains from ART for at least six months, for at least one year, for at least two years, for at least three years, for at least four years, or for at least five years after administration of the immunogenic composition.

In one embodiment, the polypeptide of the composition comprises Nef, Gag and Pol. In a further embodiment, Gag is p17, p24 or both. In another embodiment, Pol is RT. In a further embodiment, the polypeptide comprises SEQ ID NO:8. In another embodiment the immunogenic composition further comprises Env.

In one embodiment, the adjuvant of the composition is one or more components selected from: an immunologically active saponin fraction, a lipopolysaccharide, an immunostimulatory oligonucleotide, and a sterol. In a further embodiment, the adjuvant comprises an immunologically active saponin fraction and a lipopolysaccharide. In a further embodiment, the adjuvant comprises QS21 and/or a lipid A derivative. In a further embodiment, the lipid A derivative is 3D-MPL. In another embodiment the adjuvant comprises CpG. In another embodiment, the sterol is cholesterol. In a further embodiment, the adjuvant further comprises a liposome carrier.

In one embodiment, the composition is administered once to the subject. In a further embodiment, composition is administered two or more times to the subject. In a further embodiment, composition is administered as either the priming dose or boosting dose of a prime-boost regimen.

### BRIEF DESCRIPTION OF THE DRAWINGS

FIG. 1 is a graph showing the correlation between the progression of HIV disease with two major clinical markers, CD4+ T cell counts (cells/mm³) and viral load (plasma viremia dilutional titers).

FIG. 2 is a schematic of the patient cohorts described in the Examples.

FIGS. 3A-B show the CD4 count change from baseline as a function of days since administration of the first dose in the ART-experienced cohort (panel A) and ART-naïve cohort (panel B). For each data pair of each panel, the F4/AS01 is presented on the left (black) and placebo on the right (red).

FIGS. 4A-B show the viral load of the ART-naïve cohort during the study. Panel A shows the viral load change from baseline (expressed in log 10) as a function of the number of days since administration of the first dose. Placebo is the top line (grey) and F4/AS01 is the bottom line (orange). Panel B shows the reverse cumulative distribution curves (RCC) for change in log₁₀ viral load at day 44, month 4, month 7 and month 12 in the ART-naïve cohort (total vaccinated cohort). Y-axis corresponds to the percentage of subjects with a viral load change greater or equal to the corresponding value in the X-axis. Each drop in the curve corresponds to the change observed in viral load for a given subject (each dot corresponding to one subject. For each graph, F4/AS01 is represented in the line on the left (grey) and placebo on the right (thick orange line)).

FIGS. 5A-C show the percentage of F4-specific CD4⁺ CD40L⁺ T-cells expressing at least IL-2 (according to protocol cohort for immunogenicity); (A) overall response to F4 in ART-experienced and ART-naïve subjects; (B) response to specific antigens in ART-experienced subjects; and (C) response to specific antigens in ART-naïve subjects. P-values are based on 95% CI for the geometric mean ratio F4/AS01 over placebo derived by ANCOVA model adjusted for baseline (except at the pre-vaccination timepoint where no adjustment was performed [ANOVA]). For each graph, the placebo group is represented on the left (grey) and F4/AS01 is on the right (orange).

FIG. 6 is a table of results for CD4⁺ T-cell response to the F4/AS01 vaccine: Responder rates. T-cell responses were evaluated by intracellular cytokine staining after stimulation with Nef, p17, p24 and reverse transcriptase (RT) peptide pools. Results were expressed as the percentage of total CD40L⁺ CD4⁺ T-cells expressing at least IL-2. If no cytokine secretion was detectable pre-vaccination, a subject was considered a responder if the proportion of CD40L⁺CD4⁺ T-cells expressing at least IL-2 was greater than or equal to the cut-off value of 0.03%. If cytokine secretion was detectable pre-vaccination, a subject was considered a responder if the proportion of CD40L⁺CD4⁺ T-cells expressing at least IL-2 was at least two-fold higher than baseline.

FIGS. 7A-B show the cytokine co-expression profile of F4-specific CD40L⁺ CD4⁺ T-cells for (A) pre-vaccination (presented on the left of each data pair in light yellow) and two weeks post-dose 2 (day 44, presented on the right of each data pair in darker orange) in vaccinated ART-experienced patients, with (B) pie charts for all time-points. Results are expressed as the percentage of F4-specific CD40L⁺ CD4⁺ T-cells expressing 1, 2 or 3 cytokines (IL-2, TNF-α or IFN-γ).

FIGS. 8A-D show the CD8+ T cell response after administration of F4co. Panel A shows the percentage of F4-specific CD8+ T cells in the ART-experienced cohort expressing at least one marker selected from CD40L, IL-2, TNFα and/or IFNγ. Panel B shows the percentage of F4-specific CD8+ T cells in the ART-naive cohort expressing at least one marker selected from CD40L, IL-2, TNFα and/or IFNγ. Panel C shows the percentage of F4-specific CD8+ T cells in the ART-experienced cohort expressing each of the markers selected from CD40L, IL-2, TNFα and/or IFNγ. Panel D shows the percentage of F4-specific CD8+ T cells in the ART-naïve cohort expressing each of the markers selected from CD40L, IL-2, TNFα and/or IFNγ.

FIG. 9 shows the association between viral load and the frequency of F4-specific CD4⁺ CD40L⁺ T-cells expressing at least IL-2 at two weeks post-dose 2 in the ART-naïve cohort (total vaccinated cohort). Each point corresponds to data from a given subject.

FIGS. 10A-B show the humoral response to F4co for twelve months after administration in the ART-experienced cohort collectively (panel A) and by antigen (panel B). For all graphs, F4/AS01 is represented by the top line (orange) and placebo on the bottom line (grey).

FIGS. 11A-B show the humoral response to F4co for twelve months after administration in the ART-naïve cohort collectively (panel A) and by antigen (panel B). For panel A and p24 of panel B, the top line represents F4/AS01 (orange) and the bottom line represents placebo (grey). For RT, Nef and p17, the top line represents placebo (grey) and the bottom line represents F4/AS01 (orange).

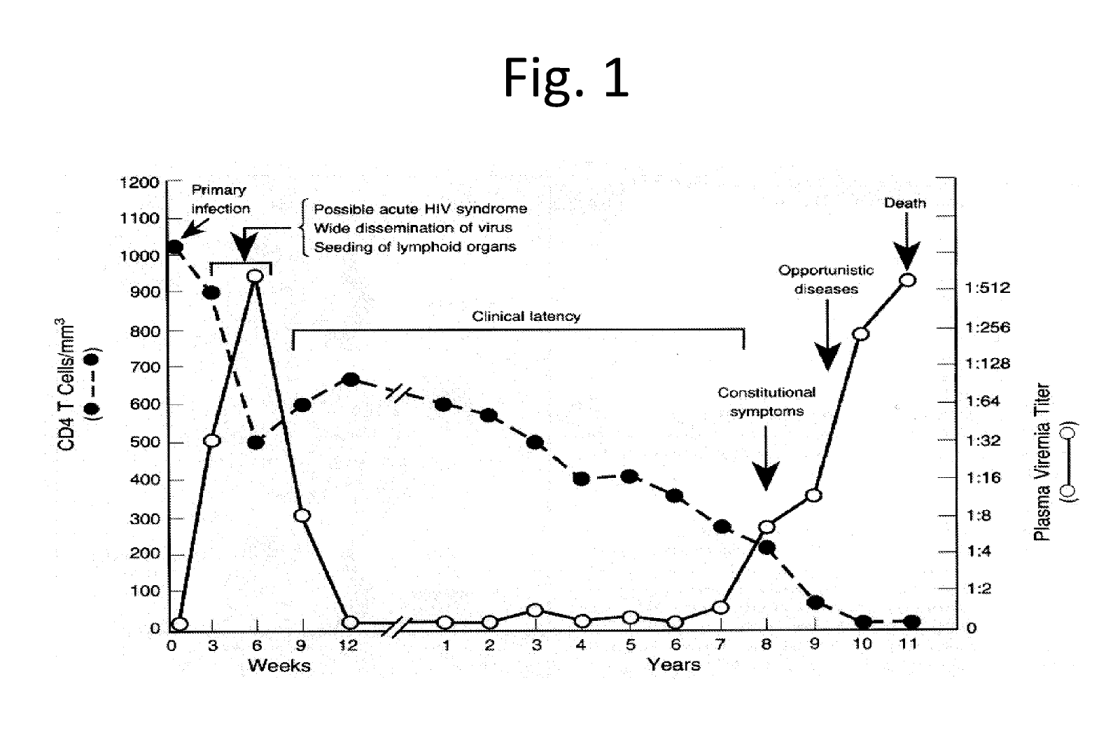

### DETAILED DESCRIPTION

Natural history studies suggest that CD8+ T-cell responses play an important role in the control of primary viremia [Borfrow, 1994] in the evolution of disease [Harrer, 1996]. CD8+ T-cells are the main immune effector mechanism for the elimination of virus-infected cells. It has been demonstrated in the SIV monkey model that experimental depletion of CD8+ T-cells leads to the loss of control of an established virus infection [Jin, 1999]. Therefore, it is one object of the present invention to prevent the depletion of CD8+ T cells in a HIV-1 infected subject.

CD4+ T-cells appear to play a role in maintaining CD8+ T-cell responses. CD4+ T-cell help is required to prime and efficiently differentiate effector and memory CD8+ T cells [Janssen, 2003; Sun, 2004]. The loss of HIV-1-specific CD8+ T-cell proliferation after acute HIV1 infection can be restored in vitro by addition of autologous CD4+ T-cells isolated during the acute infection and, importantly in vivo by vaccine-induced HIV-1-specific CD4+ T-cells [Lichterfield, 2004].

Recent reports indicate that polyfunctional HIV-specific CD4+ T-cells are associated with the long-term nonprogressor phenotype (LTNP), i.e., infected subjects who remain in the chronic, asymptomatic phase without ART [Potter, 2007; Kannanganat, 2007]. LTNP are often viral controllers, i.e., infected subjects whose viral loads remain low without intervention, and these low viral loads usually correlate with high CD4+ T cell counts. For example, studies in LTNP correlate higher levels of CD4+ T cells expressing at least two or three cytokines with non-progressing disease and low viral load [Kannanganat, 2007; Boaz, 2002; Harari, 2004 Iyasaere, 2003]. Further, memory CD4+ T cells (IL-2+) have a protective potential in HIV infection [Younes, 2003], and a strong proliferative response of these cells correlate with low viral loads [Lichterfield, 2004]. Therefore, induction of polyfunctional CD4+ T cell response can play an important role in CD8+ T cell activity and viral load management.

It has been surprisingly found that administration of the vaccine of the present invention induces high levels of polyfunctional CD4+ T cells in both ART-experienced and ART-naïve HIV-1 infected subjects. Furthermore, it has also been found administration of the present vaccine also leads to lower viral loads as compared to HIV-1 infected subjects administered a placebo. Without being limited to this hypothesis, it is believed that there is a correlation between the polyfunctional CD4+ T cell response induced in the present methods leads to a reduction in viral loads in HIV-1 infected subjects.

Therefore, it is an object of the present invention to provide methods and immunogenic compositions that induce CD4+ T cells that specifically recognize at least one polypeptide of the composition after administration to a subject infected with HIV-1. For example, after administration, the subject has increased levels of CD4+ T cells that recognize Gag, Pol, and/or Nef as compared to before administration. Suitably, the increase in response, or “higher” response is either de novo (any increase when there is no pre-existing response before administration) or a significant increase of a pre-existing response after administration, such as a two-fold or greater increase or a statistically significant increase.

Subjects that have these increased CD4+ response levels are considered “responders”, and it is an object of the invention that the immunogenic composition induces such a response in at least 20% of subjects infected with HIV-1. Suitably, at least 30%, at least 40%, at least 50%, at least 60%, at least 70%, at least 80%, at least 90%, at least 95% or 100% of subjects are responders after administration of the immunogenic composition of the present invention. In one embodiment, at least 75% of subjects are responders.

It is a further object of the invention to provide methods and immunogenic compositions to induce the expression of activation markers in CD4+ T cells after administration to a subject infected with HIV-1. For example, the CD4+ T cells express activation markers consistent with those expressed in the CD4+ T cells of long term nonprogressors and viral controllers.

In one embodiment, the CD4+ T cell activation markers include CD40L, IL-2, TNFα and IFNγ. Suitably, one, two, three or all four markers are expressed.

Also provided herein are methods and immunogenic compositions for restoring or inhibiting the loss of CD8+ T cell function, such as activation of HIV-1 specific CD8+ T cells in subjects infected with HIV-1. For example, administration of the present immunogenic compositions directly activates a CD8+ T cell response, as measured by HIV-1 specific antigen recognition, increased proliferation, increased persistence, and/or expression of activation markers.

By “antigen” it is meant a substance that upon administration to a subject triggers a specific immune response against the antigen. For example, a HIV antigen is a HIV protein, derivative or fragment thereof that triggers an immune response specific for that HIV protein, derivative or fragment thereof. In one embodiment, the antigen may be a polynucleotide encoding a protein, for example a HIV protein, derivative or fragment thereof.

Alternatively or in addition to a direct CD8+ T cell response, the immunogenic composition of the present invention can induce a CD4+ T cell response which restores or inhibits the loss of CD8+ T cell function. Such a CD4+ T cell response is described above.

The present invention also provides methods and compositions for reducing or inhibiting the expected increase in the viral load of a subject infected with HIV-1. Without being limited by the theory, it is believed the immunogenic compositions of the present invention induce an immune response, such as a CD4+ T cell or CD8+ T cell response as described above, that inhibits at least one stage of the HIV-1 life cycle such that viral loads do not increase over time as expected. Because viral load is closely associated with disease progression [Mellors et al., 1996; Fraser et al, 2007], stabilising or reducing the viral load leads to maintaining the health of the infected subject, as seen in long term nonprogressors.

Therefore, it is an object of this invention to stabilise or reduce the viral load of a subject infected with HIV-1 by administering the immunogenic composition described herein. In one embodiment, the viral load of the subject increases less than expected as compared to a similar population of infected subjects. In another embodiment, the viral load of the subject is stabilised, i.e., does not significantly increase from the viral load at the time of administration. In another embodiment, the viral load decreases after administration.

By “stabilise” it is meant that the viral load of a subject does not vary more than 5% of the viral load just prior to administration of the compositions of the claimed invention. By “reduce” it is meant that the viral load of a subject is more than 5% lower than the viral load just prior to administration of the compositions of the claimed invention. For example, reduce can mean 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% or 99.5% lower than the viral load of a subject just prior to administration of the compositions of the claimed invention.

The present invention also provides methods and compositions for preventing the progression of HIV disease in a subject infected with HIV-1 by inducing an immune response that prevents the viral load of the subject from increasing above 100,000 copies/ml. Viral loads above 100,000 copies/ml are associated with the progression of HIV disease, while viral loads between 10,000-99,999 have a lower risk, preferably less than 30,000 copies/ml. A viral load of less than 10,000 copies/ml, 1000 copies/ml or 500 copies/ml is desirable. “Determining Risk of Disease Progression”, 2007, available at aidsetc.org/aidsetc?page=cm-106_cd4_stage, last accessed on 23 Sep. 2010.

It is desirable that the suppression of viral load described above persist for a clinically significant time, such as from one month to ten or more years. In one embodiment, the suppression persists for at least four months. In further embodiments, the suppression persists for at least six months, at least twelve months, at least eighteen months, at least two years, at least three years, at least four years, at least five years, at least six years, at least seven years, at least eight years, at least nine years, or at least ten years.

Viral load, including reservoir (integrated) viral load, can be measured using a number of different assays, including numerous commercially available assays. These assays include quantitative PCR assays, branched-chain DNA assays and blood spot assays. Alternative assays that quantitate the amount of HIV present in an infected subject can also be substituted. These assays include p24 antigen assays and reverse transcriptase assays. Any suitable testing method can be used to quantitate the amount of HIV in the infected subject.

For any of the methods or assays described herein, the assays provided in the Examples can be used.

It is a further object of the present invention to provide methods and immunogenic compositions for the prevention of HIV disease progression in a subject infected with HIV-1 in the absence of antiretroviral therapy (ART). While ART has been relatively effective in managing HIV disease, such treatment has significant limitations, including drug resistance, serious side effects, compliance difficulties, cost and availability, particularly in developing countries. Therefore, long term management that overcomes these limitations remains desirable.

By “antiretroviral therapy” (ART), it is meant any of the therapies used to manage progression of HIV-1 disease, for example nucleoside and non-nucleoside reverse transcriptase inhibitors, protease inhibitors, fusion inhibitors, entry inhibitors, maturation inhibitors, cellular inhibitors and integrase strand transfer inhibitors. Such drugs include lamivudine and zidovudine, emtricitabine (FTC), zidovudine (ZDV), azidothymidine (AZT), lamivudine (3TC), zalcitabine, dideoxycytidine (ddC), tenofovir disoproxil fumarate (TDF), didanosine (ddI), stavudine (d4T), abacavir sulfate (ABC), etravirine, delavirdine (DLV), efavirenz (EFV), nevirapine (NVP), amprenavir (APV), tipranavir (TPV), indinavir (IDV), saquinavir, saquinavir mesylate (SQV), lopinavir (LPV), ritonavir (RTV), fosamprenavir calcium (FOS-APV), ritonavir, RTV, darunavir, atazanavir sulfate (ATV), nelfinavir mesylate (NFV), enfuvirtide, T-20, maraviroc and raltegravir. ART drugs can also include antibodies, such as ibalizumab, targeting HIV proteins or cellular proteins associated with disease progression. Also included are immune-based therapies, such as IL-2, IL-12 and alpha-epibromide. Each of these drugs can be administered alone or in combination with any other ART drug. Information about ART drugs and their administration can be found many pharmacopeia, such as the United States Pharmacopeia (USP) or accessed online, such as at www.aidsmeds.com (accessed 23 Sep. 2010).

In one embodiment, the viral load of the subject remains stable or declines as described above in the absence of ART after administration of the immunogenic composition of the present invention. In another embodiment, administration of the immunogenic composition induces a CD4+ T cell response that is specific for HIV as described above. In another embodiment, administration of the immunogenic composition induces expression of activation markers in CD4+ T cells as described above. In another embodiment, the administration of the immunogenic composition induces a CD8+ T cell response that is specific for HIV as described above. In another embodiment, administration of the immunogenic composition restores or inhibits the loss of CD8+ T cell function as described above. Suitably, any or all of the above embodiments can be combined.

In an embodiment, after administration, the HIV-1 disease status of the subject does not progress to clinical disease in the absence of ART. Clinical disease can be monitored in a variety of ways, such as by measuring the subject's viral load and/or CD4+ T cell counts, as well as the manifestation of disease pathologies, such as HIV-associated proliferative disorders and/or opportunistic infections. In a further embodiment, the HIV-1 disease status of the subject does not progress to the threshold recommended for initiation of ART, such as a viral load of greater than 10,000 copies/ml and/or a CD4+ T cell count of less than 500 cells/mm³, less than 350 cells/mm³ or less than 200 cells/mm³.

In one embodiment of the present invention, the subject is ART-naïve. These subjects have no prior or concurrent treatment with ART. In another embodiment, the subject is ART-experienced. These subjects can have been treated with ART prior to but not concurrently with administration of the immunogenic composition of the present invention (ART interruption). In some embodiments, the subject is concurrently treated with ART. By concurrently, it is not meant to require that the treatment with ART must occur simultaneously with administration of the claimed composition; rather it is meant that the subject is meant to be following an ART treatment regime at the time of administration, regardless of strict compliance.

It is an object of the present invention to delay the need for ART by an HIV-1 infected subject as compared to a similar population of infected subjects. In one embodiment, ART treatment is delayed for at least four months. In further embodiments, ART treatment is delayed for at least six months, at least twelve months, at least eighteen months, at least two years, at least three years, at least four years, at least five years, at least six years, at least seven years, at least eight years, at least nine years, or at least ten years. This delay can be for initial ART treatment of ART naïve subjects or can be for resumption of ART treatment in ART-experienced subjects. In one embodiment, the subject can alternate ART treatment and treatment according to the claimed methods and compositions so as to cycle between the treatment modalities.

It is a further object of the invention to provide compositions for the treatment of HIV-1 infected subjects in need thereof, such as subjects having a particular CD4+ T cell count or profile, a particular CD8+ T cell count or load, a particular viral load, a particular treatment status, or any parameter conducive to the selection of a suitable subject. For example, a subject can be selected on the basis of being ART-naïve, ART-experienced, ART-refractory and/or ART-noncompliant.

It is another object of the invention to reduce the development of ART-resistant HIV strains through the reduction of viral replication due to viral escape and/or ART-noncompliance. By providing a different modality of viral control, such development of ART-resistant HIV can be avoided.

### Immunogenic Compositions

Immunogenic compositions suitable for use in the context of the methods are disclosed herein.

The HIV-1 genome encodes a number of different proteins, each of which can be immunogenic in its entirety or as a fragment. Envelope proteins include gp120, gp41 and Env precursor gp160, for example. Non-envelope proteins of HIV-1 include for example internal structural proteins such as the products of the gag and pol genes and other non-structural proteins such as Rev, Nef, Vif and Tat.

Because CD4+ T cell responses are important for immunological control of HIV-1 progression, an HIV-1 antigen can contain at least one, and preferably more than one CD4+ T cell epitopes. The viral antigens containing the highest number of conserved T-cell epitopes are Gag, Pol, and Nef. Alternatively, or in addition to the CD4+ T cell epitopes, the antigen can contain B cell epitopes, such as provide in the Env polypeptide, and/or CD8+ T cell epitopes.

The immunogenic compositions of the invention comprise one or more of these antigens. These antigens can be combined in one or more fusion polypeptides or can be provided separately or as a mixture thereof.

In an embodiment, the immunogenic composition of the invention comprises one or more polypeptides comprising Nef.

HIV-1 Nef is an early protein, i.e. it is expressed early in infection and in the absence of structural protein. The Nef gene encodes an early accessory HIV-1 protein which has been shown to possess several activities. For example, the Nef protein is known to cause the down regulation of CD4, the HIV-1 receptor, and MHC class I molecules from the cell surface, although the biological importance of these functions is debated. Additionally Nef interacts with the signal pathways of T cells and induces an active state, which in turn can promote more efficient gene expression. Some HIV-1 isolates have mutations in this region, which cause them not to encode functional protein and are severely compromised in their replication and pathogenesis in vivo.

References to Nef are to full length Nef and to fragments, variants and derivatives of full length Nef. The term also includes polypeptides comprising Nef, including polypeptides comprising fragments, variants and derivatives of Nef.

In an embodiment the immunogenic composition of the invention comprises one or more polypeptides comprising Pol.

The Pol gene encodes two proteins containing the two activities needed by the virus in early infection, the RT and the integrase protein needed for integration of viral DNA into cell DNA. The primary product of Pol is cleaved by the virion protease to yield the amino terminal RT peptide which contains activities necessary for DNA synthesis (RNA and DNA-dependent DNA polymerase activity as well as an RNase H function) and carboxy terminal integrase protein. RT is thus an example of a fragment of Pol. HIV-1 RT is a heterodimer of full-length RT (p66) and a cleavage product (p51) lacking the carboxy terminal RNase H domain, each of which are also examples of fragments of Pol.

References to Pol are to full length Pol and to fragments, variants and derivatives of full length Pol. The term also includes polypeptides comprising Pol, including polypeptides comprising fragments, variants and derivatives of Pol.

In an embodiment, Pol comprises the RT fragment. The RT fragment is an example of a fragment of Pol. References to RT are also to full length RT and to fragments, variants and derivatives of full length RT. The term also includes polypeptides comprising RT, including polypeptides comprising fragments, variants and derivatives of RT. In this manner, RT can comprise the p66 fragment, the p51 fragment and/or fragments, variants and derivatives of p66 and/or p51.

In an embodiment the immunogenic composition of the invention comprises one or more polypeptides comprising Gag.

The Gag gene is translated as a precursor polyprotein that is cleaved by protease to yield products that include the matrix protein (p17), the capsid (p24), the nucleocapsid (p9), p6 and two space peptides, p2 and p1, all of which are examples of fragments of Gag.

The Gag gene gives rise to the 55-kilodalton (kD) Gag precursor protein, also called p55, which is expressed from the unspliced viral mRNA. During translation, the N terminus of p55 is myristoylated, triggering its association with the cytoplasmic aspect of cell membranes. The membrane-associated Gag polyprotein recruits two copies of the viral genomic RNA along with other viral and cellular proteins that triggers the budding of the viral particle from the surface of an infected cell. After budding, p55 is cleaved by the virally encoded protease (a product of the pol gene) during the process of viral maturation into four smaller proteins designated MA (matrix [p17]), CA (capsid [p24]), NC (nucleocapsid [p9]), and p6, all of which are examples of fragments of Gag.

The p17 (MA) polypeptide is from the N-terminal, myristoylated end of p55. Most MA molecules remain attached to the inner surface of the virion lipid bilayer, stabilizing the particle. A subset of MA is recruited inside the deeper layers of the virion where it becomes part of the complex which escorts the viral DNA to the nucleus. These MA molecules facilitate the nuclear transport of the viral genome because a karyophilic signal on MA is recognized by the cellular nuclear import machinery. This phenomenon allows HIV-1 to infect non-dividing cells, an unusual property for a retrovirus.

The p24 (CA) protein forms the conical core of viral particles. Cyclophilin A has been demonstrated to interact with the p24 region of p55 leading to its incorporation into HIV-1 particles. The interaction between Gag and cyclophilin A is essential because the disruption of this interaction by cyclosporin A inhibits viral replication.

The NC region of Gag is responsible for specifically recognizing the so-called packaging signal of HIV-1. The packaging signal consists of four stem loop structures located near the 5′ end of the viral RNA, and is sufficient to mediate the incorporation of a heterologous RNA into HIV-1 virions. NC binds to the packaging signal through interactions mediated by two zinc-finger motifs. NC also facilitates reverse transcription.

The p6 polypeptide region mediates interactions between p55 Gag and the accessory protein Vpr, leading to the incorporation of Vpr into assembling virions. The p6 region also contains a so-called late domain which is required for the efficient release of budding virions from an infected cell.

References to Gag are to full length Gag and to fragments, variants and derivatives of full length Gag. The term also includes polypeptides comprising Gag, including polypeptides comprising fragments, variants and derivatives of Gag.

In an embodiment, Gag comprises the p55 precursor protein. References to p55 are also to full length p55 and to fragments, variants and derivatives of full length p55. The term also includes polypeptides comprising p55, including polypeptides comprising fragments, variants and derivatives of p55. In this manner, p55 can comprise the p17 fragment, the p24 fragment, the p9 fragment, the p6 fragment and/or fragments, variants and derivatives of p17, p24, p9 and/or p6, and polypeptides comprising said fragments, variants or derivatives.

In an embodiment, Gag is p17. In an embodiment, Gag is p24. In an embodiment, Gag comprises both p17 and p24 either as separate protein antigen components or fused together.

Suitably, p17 and p24 are fused together and are separated by a heterologous amino-acid sequence.

In one embodiment, the immunogenic composition further comprises the HIV envelope protein (Env) or a fragment or a derivative thereof.

In the present invention, antigens described are full length antigens, for example, full length Nef, full length Pol, full length Gag. The invention also encompasses antigens that are not full length, including fragments or variants of the antigen, which can or can not correspond to full length. Suitably, fragments are immunogenic fragments and variants are immunogenic variants.

Typically, “fragments”, whether immunogenic or otherwise, contain a contiguous sequence of amino acids from the polypeptide comprising an HIV-1 antigen of which they are a fragment. Suitably, the fragments contain at least 5 to 8 amino acids, at least 9 to 15 amino acids, at least 20, at least 50, or at least 100 contiguous amino acids from the polypeptide of which they are a fragment.

“Immunogenic fragments”, as used herein, will comprise at least one T cell epitope or B cell epitope of the antigen and display HIV-1 antigenicity. Such fragments are capable of inducing an immune response against the native antigen, either in isolation or when presented in a suitable construct, such as when fused to other HIV-1 epitopes or antigens, fused to a fusion partner which can be proteinaceous and/or immunogenic, or when presented on or in a carrier.

The term “variant”, as used herein, includes polypeptides that have been altered in a limited way compared to their non-variant counterparts. This includes point mutations which can change the properties of the polypeptide for example by improving expression in expression systems or removing undesirable activity including undesirable enzyme activity. However, the polypeptide variant comprising an HIV-1 antigen must remain sufficiently similar to the native polypeptide such that they retain the antigenic properties desirable in an immunogenic composition or vaccine and thus they remain capable of raising an immune response against the native antigen. Whether or not a particular variant raises such an immune response can be measured by a suitable immunological assay such as an ELISA (for antibody responses) or flow cytometry using suitable staining for cellular markers and cytokines (for cellular responses).

Suitably, “variants” according to the present invention, comprise additions, deletions or substitutions of one or more amino acids. They encompass truncated antigens, where the C-terminus and/or the N-terminus of the antigen has been cleaved of one or more amino acids.

Suitably, “variants” include truncates wherein 1 to 5 amino acids, 6 to 10 amino acids, 11 to 15 amino acids, 16 to 20 amino acids, 21 to 25 amino acids or more than 25 amino acids are cleaved from the C-terminus and/or the N-terminus of the antigen

Variants of the invention can incorporate one or more deletions, additions or substitutions of one or more amino acids. Accordingly, a truncate of an antigen can additionally comprise deletions, additions or substitutions of one or more amino acids at a different part of the peptide.

Variants of the invention also comprise a polypeptide sequences that have at least 70%, 80%, 90%, 95%, 98%, 99% or 100% identity with the polypeptide sequence of Nef, Pol and/or Gag.

In an embodiment of the invention, the immunogenic compositions comprise two polypeptides comprising one or more antigens, three polypeptides comprising one or more antigens, four polypeptides comprising one or more antigens or five or more polypeptides comprising one or more antigens.

Each of Nef, Pol and/or Gag can be present in the immunogenic composition more than once. For example, an immunogenic composition of the invention can comprise two or more polypeptides comprising Nef, two or more polypeptides comprising Pol and/or two or more polypeptides comprising Gag.

In a further embodiment, each of the one or more polypeptides can comprise one of Nef, Pol and/or Gag, two of Nef, Pol and/or Gag, three of Nef, Pol and/or Gag, four of Nef, Pol and/or Gag, and so forth. If more than one polypeptide is present in the composition, each polypeptide can comprise the same number and/or composition of antigens or each polypeptide can comprise a different number and/or composition of antigens. If there are three or more polypeptides in the composition, two or more polypeptides can comprise the same number and/or composition of antigens while the remaining polypeptide(s) can comprise a different number and/or composition of antigens.

It has been well documented that the polypeptide sequences for these antigens are well conserved across different strains, including across strains from different clades of HIV-1. In an embodiment, the polypeptides of the composition is from an HIV-1 strain of clade A, B, C, D, E, F, G, H, J, K, or a circulating recombinant form of HIV-1 (CRF). In an embodiment, the polypeptides are from the same clade, such as clade B. In another embodiment, the polypeptides are from two or more clades. In a further embodiment, the polypeptides are from the same clade as the strain of HIV-1 infecting the subject. In another embodiment, at least one polypeptide is from a different clade as the strain of HIV-1 infecting the subject. In a further embodiment, all polypeptides are from a clade different from the strain of HIV-1 infecting the subject.

Reference sequences for each HIV-1 clade and strain is readily available in various well-known genetic databases, such as Genbank accessible at www.ncbi.nlm.nih.gov/genbank/ or the UniProt database accessible at www.uniprot.org/ (both accessed on 21 Sep. 2011). For example, sequences for each antigen of the invention for each clade and/or strain can be found in these databases.

Fusion proteins comprising one or more of the antigens which can be present in the immunogenic composition of the invention have been disclosed in WO2006/013106, incorporated herein by reference. The antigens Pol, Nef, Gag and variants and fragments thereof have previously been selected for inclusion in a fusion protein for use in an immunogenic composition because they are considered to be relatively well conserved across different strains of HIV, and thus should be more likely to cross-react with antigens from different strains of HIV, than less well conserved antigens. However, the incorporation of these antigens into fusion proteins can introduce unpredictable complications because the antigens therein do not correspond to native proteins. Accordingly, fusion proteins are not straightforward to produce and cannot be presumed to behave as the native protein would.

In an embodiment of the invention, two, three, four or more of the antigens in the immunogenic composition are fused to form a fusion protein.

Suitably, Gag is fused to Pol or Pol is fused to Gag, Pol is fused to Nef or Nef is fused to Pol, and/or Nef is fused to Gag or Gag is fused to Nef.

Suitably, in a fusion protein of the invention, Gag is p17 and/or p24, and/or Pol is RT.

In particular, it is convenient that the antigens in the immunogenic composition are fused to form a fusion protein comprising Nef, RT, p17 and p24 in any order. Suitably, the antigens are fused to form a fusion protein comprising p24-RT-Nef-p17. Such a fusion protein is known as F4.

The antigens in a fusion protein can be fused directly to each other or by means of a linker. Such linker can comprise a heterologous amino acid sequence comprising one or more amino acids.

The antigens in the fusion can be from the same strain of HIV, can be from different strains within the same HIV-1 clade or can be from different strains from different HIV-1 clades.

Suitably, the antigens in the fusion protein are from HIV-1 strains from two, three or four different HIV-1 clades. Alternatively, all of the antigens in the fusion protein are from an HIV-1 strain or strains from the same HIV-1 clade.

The peptides according to the invention can be combined with other antigens. In particular, this can include HIV-1 env proteins or fragments or variants thereof. Preferred forms of env are gp120, gp140, gp41 and gp160. The env can be for example the envelope protein described in WO 00/07631 from an HIV-1 clade B envelope clone known as R2, or fragments or variants thereof. The env can also be the gp120 clone known as W61.D, or fragments or variants thereof.

Thus the invention further provides an immunogenic composition according to the invention further comprising an HIV-1 env protein or fragment or variant thereof. For the sake of clarity, the meaning of the terms “fragment” and “variant” used here are as defined above.

In an embodiment, immunogenic compositions of the invention that comprise a fusion protein further comprise one or more unfused polypeptides comprising an antigen.

Suitably, the antigen in the unfused polypeptide is from a strain of HIV-1 from the same clade as at least one of the antigens in the fusion protein.

Alternatively, the antigen in the unfused polypeptide is from a strain of HIV-1 different from the one or more clades in the fusion protein.

Suitably, the unfused polypeptide comprises Env. For instance, the unfused polypeptide comprises one or more of gp120, gp140 or gp160.

The HIV-1 envelope glycoprotein gp120 is the viral protein that is used for attachment to the host cell. The gp120 protein is the principal target of neutralizing antibodies, as well as antibody-dependent cellular cytotoxicity (ADCC) and antibody-dependent cell-mediated viral inhibition (ADCVI). However, the regions of the protein most commonly recognised by antibodies (V3 loop) are also the most variable parts of the protein. The gp120 protein also contains epitopes that are recognized by cytotoxic T lymphocytes (CTL). These effector cells are able to eliminate virus-infected cells, and therefore constitute a second major antiviral immune mechanism. In contrast to the target regions of neutralizing antibodies some CTL epitopes appear to be relatively conserved among different HIV-1 strains. For this reason gp41, gp120 and gp160 can be useful antigenic components in vaccines that aim at eliciting cell-mediated immune responses (particularly CTL).

The immunogenic compositions, or vaccines, of the present invention will contain an immunoprotective or immunotherapeutic quantity of the polypeptide and can be prepared by conventional techniques.

In an embodiment, the total amount of each antigen in a single dose of the immunogenic composition is 0.5-25 μg, 2-20 μg, 5-15 μg, or around 10 μg.

Suitably, the total amount of fusion protein in a single dose of the immunogenic composition is 10 μg and/or the total amount of unfused polypeptide in a single dose of the immunogenic composition is 20 μg.

In an embodiment, the total amount of all antigens in a single dose of the immunogenic composition is 0.1 μg-100 μg, 0.5-50 μg, 2-40 μg, 5-30 μg, 7-20 μg, For example, the total amount of all antigens can be about 100 μg, about 90 μg, about 80 μg, about 70 μg, about 60 μg, about 50 μg, about 40 μg, 30 μg, about 20 μg or about 10 μg.

The amount of protein in a dose of the immunogenic composition is selected as an amount which induces an immune response without significant, adverse side effects in typical recipients. Such amount will vary depending upon which specific immunogen is employed and the dosing or vaccination regimen that is selected. An optimal amount for a particular immunogenic composition can be ascertained by standard studies involving observation of relevant immune responses in subjects.

### Adjuvants

Adjuvants are described in general in Vaccine Design—the Subunit and Adjuvant Approach, edited by Powell and Newman, Plenum Press, New York, 1995, incorporated herein by reference.

An adjuvant, an example of an immunostimulant, refers to the components in an immunogenic composition that enhance or potentiate a specific immune response (antibody and/or cell-mediated) to an antigen.

Adjuvants can induce immune responses of the Th1-type and Th-2 type response. Th1-type cytokines (e.g., IFN-γ, IL-2, and IL-12) tend to favour the induction of cell-mediated immune response to a given antigen, while Th-2 type cytokines (e.g., IL-4, IL-5, IL-6, IL-10) tend to favour the induction of humoral immune responses to the antigen.

In the present invention, the adjuvant is a preferential inducer of a Th1 immune response.

The distinction of Th1 and Th2-type immune response is not absolute. In reality an individual will support an immune response which is described as being predominantly Th1 or predominantly Th2. However, it is often convenient to consider the families of cytokines in terms of that described in murine CD4+ T cell clones by Mosmann and Coffman, 1989, incorporated herein by reference). Traditionally, Th1-type responses are associated with the production of the INF-γ and IL-2 cytokines by T-lymphocytes. Other cytokines often directly associated with the induction of Th1-type immune responses are not produced by T-cells, such as IL-12. Suitable adjuvant systems which promote a predominantly Th1 response include monophosphoryl lipid A or a derivative thereof (or detoxified lipid A in general—see for instance U.S. Pat. Appl. Pub. No. 2008/138359, which is hereby incorporated by reference in its entirety), particularly 3-de-O-acylated monophosphoryl lipid A (3D-MPL) (for its preparation see U.S. Pat. No. 4,912,094, which is incorporated by reference in its entirety); and a combination of monophosphoryl lipid A, preferably 3-de-O-acylated monophosphoryl lipid A, together with either an aluminum salt (for instance aluminum phosphate or aluminum hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen [Thoelen et al., 1998;U.S. Pat. No. 5,776,468, each incorporated herein by reference].

An enhanced system involves the combination of a monophosphoryl lipid A and a saponin derivative, particularly the combination of QS21 and 3D-MPL as disclosed in WO 94/00153 incorporated herein by reference, or a less reactogenic composition where the QS21 is quenched with cholesterol as disclosed in U.S. Pat. No. 6,846,489, incorporated herein by reference. A particularly potent adjuvant formulation involving QS21, 3D-MPL and tocopherol in an oil in water emulsion is described in U.S. Pat. No. 6,146,632, incorporated herein by reference. In one embodiment the immunogenic composition additionally comprises a saponin, which can be QS21. The formulation can also comprise an oil in water emulsion and tocopherol (U.S. Pat. No. 6,146,632). In one embodiment, the formulation contains MF59, a squalene containing oil-in-water emulsion.

In an embodiment of the invention, the adjuvant comprises one or more components selected from an immunologically active saponin fraction and/or a lipopolysaccharide and/or an immunostimulatory oligonucleotides.

In an embodiment, the adjuvant comprises an immunologically active saponin fraction and a lipopolysaccharide.

Suitably, the immunologically active saponin fraction is QS21 and/or the lipopolysaccharide is a lipid A derivative. Suitably, the lipid A derivative is 3D-MPL.

Suitable adjuvants are combinations of 3D-MPL and QS21 (U.S. Pat. No. 5,750,110, incorporated herein by reference), oil in water emulsions comprising 3D-MPL and QS21 (U.S. Pat. No. 6,146,632, WO 98/56414), or 3D-MPL formulated with other carriers (U.S. Pat. No. 5,776,468, incorporated herein by reference).

3D-MPL is available from GlaxoSmithKline Biologicals North America and primarily promotes CD4+ T cell responses with an IFN-γ (Th1) phenotype. It can be produced according to the methods disclosed in U.S. Pat. No. 4,912,094. Chemically it is a mixture of 3-deacylated monophosphoryl lipid A with 3, 4, 5 or 6 acylated chains. Preferably in the compositions of the present invention small particle 3D-MPL is used. Small particle 3D-MPL has a particle size such that it can be sterile-filtered through a 0.22 μm filter. Such preparations are described in U.S. Pat. No. 5,776,468, incorporated herein by reference.

Another suitable adjuvant for use in the present invention is Quil A and its derivatives. Quil A is a saponin preparation isolated from the South American tree Quilaja Saponaria Molina and was first described as having adjuvant activity by Dalsgaard et al. in 1974 (“Saponin adjuvants”, Archiv. für die gesamte Virusforschung, Vol. 44, Springer Verlag, Berlin, p243-254, incorporated herein by reference). Purified fragments of Quil A have been isolated by HPLC which retain adjuvant activity without the toxicity associated with Quil A (U.S. Pat. No. 5,604,106, incorporated herein by reference), for example QS7 and QS21 (also known as QA7 and QA21). QS21 is a natural saponin derived from the bark of Quillaja saponaria Molina which induces CD8+ cytotoxic T cells (CTLs), Th1 cells and a predominant IgG2a antibody response and is a preferred saponin in the context of the present invention.

Particular formulations of QS21 have been described which are particularly suitable, these formulations further comprise a sterol (U.S. Pat. No. 6,846,489, incorporated herein by reference). The saponins forming part of the present invention can be separate in the form of micelles, mixed micelles (preferentially, but not exclusively with bile salts) or can be in the form of ISCOM matrices (U.S. Pat. No. 4,578,269, incorporated herein by reference), liposomes or related colloidal structures such as worm-like or ring-like multimeric complexes or lipidic/layered structures and lamellae when formulated with cholesterol and lipid, or in the form of an oil in water emulsion (for example as in U.S. Pat. No. 6,146,632, which is incorporated herein by reference in its entirety). The saponins can be associated with a metallic salt, such as aluminium hydroxide or aluminium phosphate (U.S. Pat. No. 6,464,489, incorporated herein by reference).

An enhanced system involves the combination of a monophosphoryl lipid A (or detoxified lipid A) and a saponin derivative, particularly the combination of QS21 and 3D-MPL as disclosed in WO 94/00153, incorporated herein by reference, or a less reactogenic composition where the QS21 is quenched with cholesterol as disclosed in U.S. Pat. No. 6,846,489. A particularly potent adjuvant formulation involving tocopherol with or without QS21 and/or 3D-MPL in an oil in water emulsion is described in U.S. Pat. No. 6,146,632.

In an embodiment, the adjuvant comprises a sterol, which can suitably be cholesterol. Suitable sterols, for instance cholesterol, act to reduce the reactogenicity of the composition while maintaining the adjuvant effect of the saponin.

In an embodiment of the invention, the adjuvant comprises a liposome carrier.

In an embodiment, the adjuvant comprises a saponin and a sterol with a ratio of saponin:sterol from 1:1 to 1:100 (w/w). Suitably, the ratio of saponin:sterol is from 1:1 to 1:10 (w/w) or the ratio of saponin:sterol is from 1:1 to 1:5 (w/w).

In an embodiment, the adjuvant comprises a saponin and a lipopolysaccharide with a ratio of saponin:lipopolysaccharide of 1:1.

Suitably, the adjuvant comprises a lipopolysaccharide and said lipopolysaccharide is present at an amount of 1-60 μg per dose. Suitably, the lipopolysaccharide is present at an amount of 50 μg per dose, 25 μg per dose, 10 μg per dose or 5 μg per dose.

Suitably, the adjuvant comprises a saponin and said saponin is present at an amount of 1-60 μg per dose. Suitably, the saponin is present at an amount of 50 μg per dose, 25 μg per dose, 10 μg per dose or 5 μg per dose.

In an embodiment, the adjuvant comprises (per 0.5 mL dose) 0.025-2.5, 0.05-1.5, 0.075-0.75, 0.1-0.3, or 0.125-0.25 mg (e.g. 0.2-0.3, 0.1-0.15, 0.25 or 0.125 mg) sterol (for instance cholesterol).

In an embodiment, the adjuvant comprises (per 0.5 mL dose) 5-60, 10-50, or 20-30 μg (e.g. 5-15, 40-50, 10, 20, 30, 40 or 50 μg) lipid A derivative (for instance 3D-MPL).

In an embodiment, the adjuvant comprises (per 0.5 mL dose) 5-60, 10-50, or 20-30 μg (e.g. 5-15, 40-50, 10, 20, 30, 40 or 50 μg) saponin (for instance QS21).

In an embodiment, the adjuvant comprises 50 μg 3D-MPL and 50 μg QS21 in a liposome-based formulation. In a further embodiment, the adjuvant comprises 25 μg 3D-MPL and 25 μg QS21 in a liposome-based formulation.

In an embodiment of the invention, the adjuvant comprises an oil-in-water emulsion.

Suitably, the oil-in-water emulsion comprises squalene and/or alpha tocopherol. Suitably, the oil-in-water emulsion is a metabolisable oil-in-water emulsion. In particular, the oil-in-water emulsion suitably comprises an emulsifier such as Tween 80.

The adjuvant can suitably comprise a saponin and a lipopolysaccharide. In particular, the adjuvant can comprise a saponin and a lipopolysaccharide at a ratio of saponin:lipopolysaccharide in the range 1:10 to 10:1 (w/w).

The adjuvant can suitably comprise a saponin and a sterol. In particular, the adjuvant can comprise a saponin and a sterol at a ratio of saponin:sterol in the range of 1:1 to 1:20 (w/w).

The adjuvant can suitably comprise a saponin and a metabolisable oil. In particular, the adjuvant can comprise a saponin and a metabolisable oil at a ratio of metabolisable oil:saponin is in the range from 1:1 to 250:1 (w/w).

The adjuvant can suitably comprise alpha tocopherol.

In an embodiment, the adjuvant comprises (per 0.5 mL dose) 0.5-15, 1-13, 2-11, 4-8, or 5-6 mg (e.g. 2-3, 5-6, or 10-11 mg) metabolisable oil (such as squalene).

In an embodiment, the adjuvant comprises (per 0.5 mL dose) 0.1-10, 0.3-8, 0.6-6, 0.9-5, 1-4, or 2-3 mg (e.g. 0.9-1.1, 2-3 or 4-5 mg) emulsifier (such as Tween 80).

In an embodiment, the adjuvant comprises (per 0.5 mL dose) 0.5-20, 1-15, 2-12, 4-10, 5-7 mg (e.g. 11-13, 5-6, or 2-3 mg) tocol (such as alpha tocopherol).

In an embodiment, the adjuvant comprises (per 0.5 mL dose) 5-60, 10-50, or 20-30 μg (e.g. 5-15, 40-50, 10, 20, 30, 40 or 50 μg) lipid A derivative (for instance 3D-MPL).

In an embodiment, the adjuvant comprises (per 0.5 mL dose) 0.025-2.5, 0.05-1.5, 0.075-0.75, 0.1-0.3, or 0.125-0.25 mg (e.g. 0.2-0.3, 0.1-0.15, 0.25 or 0.125 mg) sterol (for instance cholesterol).

In an embodiment, the adjuvant comprises (per 0.5 mL dose) 5-60, 10-50, or 20-30 μg (e.g. 5-15, 40-50, 10, 20, 30, 40 or 50 μg) saponin (for instance QS21).

In another embodiment, the adjuvant comprises a metal salt and a lipid A derivative.

Such adjuvant systems of interest include those based on aluminium salts in conjunction with the lipopolysaccharide 3-de-O-acylated monophosphoryl lipid A. The antigen and 3-de-O-acylated monophosphoryl lipid A can be co-adsorbed to the same metallic salt particles or can be adsorbed to distinct metallic salt particles.

Suitably, the adjuvant comprises (per 0.5 mL dose) 100-750, 200-500, or 300-400 μg Al, for instance as aluminium phosphate. In such embodiment, the adjuvant comprises (per 0.5 mL dose) 5-60, 10-50, or 20-30 μg (e.g. 5-15, 40-50, 10, 20, 30, 40 or 50 μg) lipid A derivative (for instance 3D-MPL).

In an embodiment of the invention, the adjuvant comprises an immunostimulatory oligonucleotide comprises a CpG motif.

Immunostimulatory oligonucleotides can be used in the immunogenic composition of the invention. The preferred oligonucleotides for use in adjuvants or immunogenic compositions of the present invention are CpG containing oligonucleotides, preferably containing two or more dinucleotide CpG motifs separated by at least three, more preferably at least six or more nucleotides. A CpG motif is a Cytosine nucleotide followed by a Guanine nucleotide. The CpG oligonucleotides of the present invention are typically deoxynucleotides. In a preferred embodiment the internucleotide in the oligonucleotide is phosphorodithioate, or more preferably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. Nos. 5,666,153 and 5,278,302 and WO95/26204, each incorporated herein by reference.

Examples of preferred oligonucleotides have the following sequences. The sequences preferably contain phosphorothioate modified internucleotide linkages.

OLIGO 1(SEQ ID NO: 1):
TCC ATG ACG TTC CTG ACG TT (CpG 1826)
OLIGO 2 (SEQ ID NO: 2):
TCT CCC AGC GTG CGC CAT (CpG 1758)
OLIGO 3(SEQ ID NO: 3):
ACC GAT GAC GTC GCC GGT GAC GGC ACC ACG
OLIGO 4 (SEQ ID NO: 4):
TCG TCG TTT TGT CGT TTT GTC GTT (CpG 2006)
OLIGO 5 (SEQ ID NO: 5):
TCC ATG ACG TTC CTG ATG CT (CpG 1668)
OLIGO 6 (SEQ ID NO: 6):
TCG ACG TTT TCG GCG CGC GCC G (CpG 5456)

Alternative CpG oligonucleotides can comprise the preferred sequences above in that they have inconsequential deletions or additions thereto.

The CpG oligonucleotides utilised in the present invention can be synthesized by any method known in the art (for example see EP 468520, incorporated herein by reference). Suitably, such oligonucleotides can be synthesized utilising an automated synthesizer.

### Administration

Administration of the pharmaceutical composition can take the form of one or of more than one individual dose, for example as repeat doses of the same polypeptide containing composition, or in a heterologous “prime-boost” vaccination regime.

In an embodiment, the immunogenic composition of the invention is initially administered to a subject as two or three doses, wherein the doses are separated by a period of one week, two weeks, three weeks, four weeks, five weeks, six weeks, eight weeks, ten weeks or twelve weeks. Suitably, the doses are separated by four weeks.

Suitably, the composition is administered to a subject (for instance as a booster) every 6-24, or 9-18 months, for instance annually. For instance, the composition is administered to a subject (for instance as a booster) at six month or 1 year intervals.

Suitably in this respect, subsequent administrations of the composition to the subject boost the immune response of earlier administrations of the composition to the same subject.

Suitably, the composition is the priming dose. Alternatively, the composition is the boosting dose.

Suitably, two or more priming and/or boosting doses are administered.

A heterologous prime-boost regime uses administration of different forms of immunogenic composition or vaccine in the prime and the boost, each of which can itself include two or more administrations. The priming composition and the boosting composition will have at least one antigen in common, although it is not necessarily an identical form of the antigen, it can be a different form of the same antigen.

Prime boost immunisations according to the invention can be homologous prime-boost regimes or heterologous prime-boost regimes. Homologous prime-boost regimes utilize the same composition for prime and boost, for instance the immunogenic composition of the invention.

Heterologous prime-boost regimes can be performed with a combination of protein and DNA-based formulations. Such a strategy is considered to be effective in inducing broad immune responses. Adjuvanted protein vaccines induce mainly antibodies and CD4+ T cell immune responses, while delivery of DNA as a plasmid or a recombinant vector induces strong CD8+ T cell responses. Thus, the combination of protein and DNA vaccination can provide for a wide variety of immune responses. This is particularly relevant in the context of HIV, since antibodies (neutralising as well as those associated with ADCC and ADCVI), CD4+ T cells and CD8+ T cells are thought to be important for the immune defense against HIV-1. For example, the DNA can be delivered in the context of an adenoviral vector.

In a further aspect of the invention, the immunogenic composition of the invention is a vaccine composition.

Vaccine preparation is generally described in New Trends and Developments in Vaccines, edited by Voller et al., University Park Press, Baltimore, Md., U.S.A. 1978, incorporated herein by reference.

Embodiments herein relating to “immunogenic compositions” of the invention are also applicable to embodiments relating to “vaccines” of the invention, and vice versa.

The terms “comprising”, “comprise” and “comprises” herein are intended by the inventors to be optionally substitutable with the terms “consisting of”, “consist of” and “consists of”, respectively, in every instance.

All references or patent applications cited within this patent specification are incorporated by reference herein.

In order that this invention can be better understood, the following examples are set forth. These examples are for purposes of illustration only, and are not to be construed as limiting the scope of the invention in any manner.

### EXAMPLES

The following examples and data illustrate the invention, but are not limiting upon the invention.

### 1. Preparation of the Exemplary F4 Candidate Vaccine

The F4 candidate vaccine was prepared as previously described in U.S. Pat. No. 7,612,173, and U.S. Appl. No. 61/181,130, which are each hereby incorporated by reference in its entirety. This vaccine contains 10 μg/dose of the codon-optimised F4 recombinant protein (F4co) adjuvanted with a proprietary adjuvant ASO1_{B}, which is liposome-based adjuvant containing the immunostimulants 50 μg each of 3-D-MPL and QS21. Alternatively, the proprietary adjuvant ASO1_{E} can be used, which contains the same immunostimulants at half the concentration of ASO1_{B}, i.e., 25 μg 3-D-MPL and 25 μg QS21. The methods are briefly summarised below.

### 2. The F4co Fusion Antigen

F4co is a fusion protein that comprises 4 HIV-1 clade B derived antigens arranged as follows: p24-RT-Nef-p17. The four antigens are:
p24, a viral capsid protein coded by the gag gene
RT (reverse transcriptase), a viral enzyme responsible for transcribing the viral RNA into double-stranded DNA. This enzyme was mutated in one amino acid (Tryptophan 229 substituted by Lysine) to remove the RT polymerase activity. This protein is coded by the pol gene
Nef, a regulatory protein coded by an open-reading-frame (ORF) that flanks the env gene
p17, a viral matrix protein coded by the gag gene.

The following polynucleotide sequence is codon optimized such that the codon usage resembles the codon usage in a highly expressed gene in E. coli without changing the amino acid sequence of the expressed fusion protein.

### 2.1 Nucleotide Sequence for F4co:

(SEQ ID NO:7)
atggtcattgttcagaacatacagggccaaatggtccaccaggcaattagtccgcgaactcttaatgcatgggtgaaggtcgt
ggaggaaaaggcattctccccggaggtcattccgatgttttctgcgctatctgagggcgcaacgccgcaagaccttaataccat
gcttaacacggtaggcgggcaccaagccgctatgcaaatgctaaaagagactataaacgaagaggccgccgaatgggatcg
agtgcacccggtgcacgccggcccaattgcaccaggccagatgcgcgagccgcgcgggtctgatattgcaggaactacgtct
acccttcaggagcagattgggtggatgactaacaatccaccaatcccggtcggagagatctataagaggtggatcatactgg
gactaaacaagatagtccgcatgtattctccgacttctatactggatatacgccaaggcccaaaggagccgttcagggactat
gtcgaccgattctataagacccttcgcgcagagcaggcatcccaggaggtcaaaaattggatgacagaaactcttttggtgca
gaatgcgaatccggattgtaaaacaattttaaaggctctaggaccggccgcaacgctagaagagatgatgacggcttgtcag

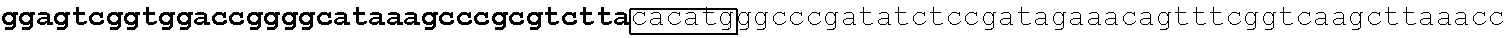

agggatggatggtccaaaggtcaagcagtggccgctaacggaagagaagattaaggcgctcgtagagatttgtactgaaatggaga
aggaaggcaagataagcaagatcgggccagagaacccgtacaatacaccggtatttgcaataaagaaaaaggattcaacaaaatgg
cgaaagcttgtagattttagggaactaaacaagcgaacccaagacttttgggaagtccaactagggatcccacatccagccggtctaa
agaagaagaaatcggtcacagtcctggatgtaggagacgcatattttagtgtaccgcttgatgaggacttccgaaagtatactgcgttta
ctataccgagcataaacaatgaaacgccaggcattcgctatcagtacaacgtgctcccgcagggctggaaggggtctccggcgatatt
tcagagctgtatgacaaaaatacttgaaccattccgaaagcagaatccggatattgtaatttaccaatacatggacgatctctatgtgggc
tcggatctagaaattgggcagcatcgcactaagattgaggaactgaggcaacatctgatcgatggggcctcactactcccgacaaga
agcaccagaaggagccgccgttcctaaagatgggctacgagatcatccggacaagtggacagtacagccgatagtgctgcccgaa
aaggattatggaccgtaaatgatattcagaaactagtcggcaagataactgggcctctcagatttacccaggcattaaggtccgacag
ctttgcaagctactgaggggaactaaggctctaacagaggtcatcccattaacggaggaagcagagcttgagctggcagagaatcgc
gaaattataaggagccggtgcacggggtatactacgacccctccaaggaccttatagccgagatccagaagcaggggcagggcca
atggacgtaccagatatatcaagaaccgtttaagaatctgaagactgggaagtacgcgcgcatgcgaggggctcatactaatgatgta
aagcaacttacggaagcagtacaaaagattactactgagtctattgtgatatggggcaagaccccaaagttcaagctgcccatacagaa
ggaaacatgggaaacatggtggactgaatattggcaagctacctggattccagaatgggaatttgtcaacacgccgccacttgttaagc
tttggtaccagcttgaaaaggagccgatagtaggggcagagaccttctatgtcgatggcgccgcgaatcgcgaaacgaagctaggca
aggcgggatacgtgactaataggggccgccaaaaggtcgtaaccatacggataccaccaatcagaagactgaactacaagcgattt
accttgcacttcaggatagtggcctagaggtcaacatagtcacggactctcaatatgcgcttggcattattcaagcgcagccagatcaaa
gcgaaagcgagcttgtaaaccaaataatagaacagatataaagaaagagaaggtatatctggcctgggtccccgctcacaagggaa

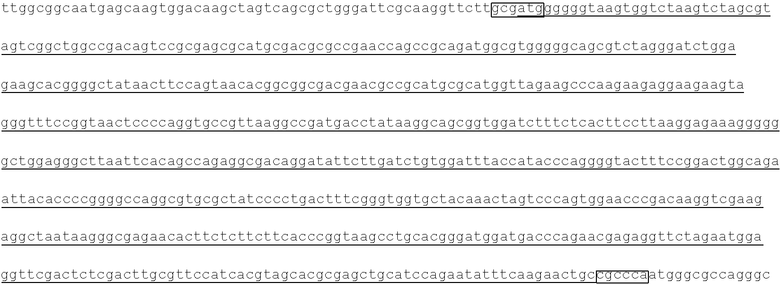

cagtgtacttagtggcggagaactagatcgatgggaaaagatacgcctacgcccggggggcaagaagaagtacaagataagcaca
ttgtgtgggcctctcgcgaacttgagcgattcgcagtgaatccaggcctgcttgagacgagtgaaggctgtaggcaaattctggggca
gctacagccgagcctacagactggcagcgaggagatcgtagtattataataccgtcgcgactctctactgcgttcatcaacgaattga
aataaaggatactaaagaggccatgataaaattgaggaggaacagaataagtcgaaaaagaaggcccagcaggccgccgccgac
accgggcacagcaaccaggtgtcccaaaactactaa
p24 sequence is in bold
Nef sequence is underlined
Boxes: nucleotides introduced by genetic construction

### 2.2 Amino Acid Sequence for F4co

(SEQ ID NO:8)

| | |
|---|---|
| MVIVQNIQGQMVHQAISPRTLNAWVKVVEEKAFSPEVIPMFSALSEGATP | 50 |
| QDLNTMLNTVGGHQAAMQMLKETINEEAAEWDRVHPVHAGPIAPGQMREP | 100 |
| RGSDIAGTTSTLQEQIGWMTNNPPIPVGEIYKRWIILGLNKIVRMYSPTS | 150 |
| ILDIRQGPKEPFRDYVDRFYKTLRAEQASQEVKNWMTETLLVQNANPDCK | 200 |
| 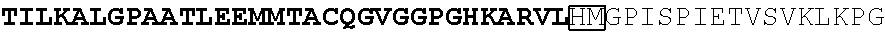 | 250 |
| MDGPKVKQWPLTEEKIKALVEICTEMEKEGKISKIGPENPYNTPVFAIKK | 300 |
| KDSTKWRKLVDFRELNKRTQDFWEVQLGIPHPAGLKKKKSVTVLDVGDAY | 350 |
| FSVPLDEDFRKYTAFTIPSINNETPGIRYQYNVLPQGWKGSPAIFQSSMT | 400 |
| KILEPFRKQNPDIVIYQYMDDLYVGSDLEIGQHRTKIEELRQHLLRWGLT | 450 |
| 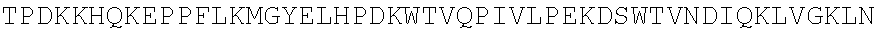 | 500 |
| WASQIYPGIKVRQLCKLLRGTKALTEVIPLTEEAELELAENREILKEPVH | 550 |
| GVYYDPSKDLIAEIQKQGQGQWTYQIYQEPFKNLKTGKYARMRGAHTNDV | 600 |
| KQLTEAVQKITTESIVIWGKTPKFKLPIQKETWETWWTEYWQATWIPEWE | 650 |
| FVNTPPLVKLWYQLEKEPIVGAETFYVDGAANRETKLGKAGYVTNRGRQK | 700 |
| VVTLTDTTNQKTELQAIYLALQDSGLEVNIVTDSQYALGIIQAQPDQSES | 750 |
| 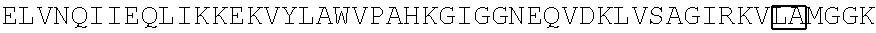 | 800 |
| WSKSSVVGWPTVRERMRRAEPAADGVGAASRDLEKHGAITSSNTAATNAA | 850 |
| CAWLEAQEEEEVGFPVTPQVPLRPMTYKAAVDLSHFLKEKGGLEGLIHSQ | 900 |
| RRQDILDLWIYHTQGYFPDWQNYTPGPGVRYPLTFGWCYKLVPVEPDKVE | 950 |
| EANKGENTSLLHPVSLHGMDDPEREVLEWRFDSRLAFHHVARELHPEYFK | 1000 |
| 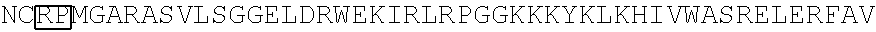 | 1050 |
| NPGLLETSEGCRQILGQLQPSLQTGSEELRSLYNTVATLYCVHQRIEIKD | 1100 |
| TKEALDKIEEEQNKSKKKAQQAAADTGHSNQVSQNY | 1136 |

P24 sequence: amino-acids 1-232 (in bold)
RT sequence: amino-acids 235-795
Nef sequence: amino-acids 798-1002
P17 sequence: amino-acids 1005-1136
Boxes: amino-acids introduced by genetic construction
K (Lysine): instead of Tryptophan (W). Mutation introduced to remover enzyme activity.

### 2.3 Alternative Sequences

Alternatively, variants of the F4co sequences can be used, for example the RT region can be mutated as follows and substituted into the construct for the indicated RT region (amino acids 235-795 of SEQ ID NO:8). The RT/p66 region between amino acids 428-448 is susceptible to E. coli proteases. The P51 construct terminates at Leu 427 resulting in the elimination of RNaseH domain. The putative E. coli “frameshift” sequences identified in RT native gene sequence were also eliminated (by codon-optimization of p51 gene).

The sequence of the synthetic P51 gene was designed according to E. coli codon usage. Thus it was codon optimized such that the codon usage resembles the codon usage in a highly expressed gene in E. coli. The synthetic gene was constructed as follows: 32 oligonucleotides were assembled in a single-step PCR. In a second PCR the full-length assembly was amplified using the ends primers and the resulting PCR product was cloned into pGEM-T intermediate plasmid. After correction of point errors introduced during gene synthesis, the p51 synthetic gene was cloned into pET29a expression plasmid. This recombinant plasmid was used to transform B834 (DE3) cells.

### P51 RT Nucleotide Sequence

[SEQ ID NO:9]

| | |
|---|---|
| 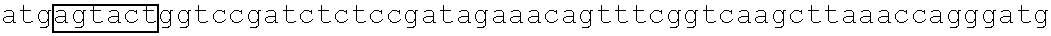 | 60 |
| gatggtccaaaggtcaagcagtggccgctaacggaagagaagattaaggcgctcgtagag | 120 |
| atttgtactgaaatggagaaggaaggcaagataagcaagatcgggccagagaacccgtac | 180 |
| aatacaccggtatttgcaataaagaagaaggattcaacaaaatggcgaaagcttgtagat | 240 |
| tttagggaactaaacaagcgaacccaagacttttgggaagtccaactaggtatcccacat | 300 |
| ccagccggtctaaagaagaagaaatcggtcacagtcctggatgtaggagacgcatatttt | 360 |
| agtgtaccgcttgatgaggacttccgaaagtatactgcgtttactataccgagcataaac | 420 |
| aatgaaacgccaggcattcgctatcagtacaacgtgctcccgcagggctggaaggggtct | 480 |
| ccggcgatatttcagagctctatgacaaaaatacttgaaccattccgaaagcagaatccg | 540 |
| gatattgtaatttaccaatacatggacgatctctatgtgggctcggatctagaaattggg | 600 |
| cagcatcgcactaagattgaggaactgaggcaacatctgcttcgatggggcctcactact | 660 |
| cccgacaagaagcaccagaaggagccgccgttcctaaagatgggctacgagcttcatccg | 720 |
| gacaagtggacagtacagccgatagtgctgcccgaaaaggattcttggaccgtaaatgat | 780 |
| attcagaaactagtcggcaagcttaactgggcctctcagatttacccaggcattaaggtc | 840 |
| cgacagctttgcaagctactgaggggaactaaggctctaacagaggtcatcccattaacg | 900 |
| gaggaagcagagcttgagctggcagagaatcgcgaaattcttaaggagccggtgcacggg | 960 |
| gtatactacgacccctccaaggaccttatagccgagatccagaagcaggggcagggccaa | 1020 |
| tggacgtaccagatatatcaagaaccgtttaagaatctgaagactgggaagtacgcgcgc | 1080 |
| atgcgaggggctcatactaatgatgtaaagcaacttacggaagcagtacaaaagattact | 1140 |
| actgagtctattgtgatatggggcaagaccccaaagttcaagctgcccatacagaaggaa | 1200 |
| acatgggaaacatggtggactgaatattggcaagctacctggattccagaatgggaattt | 1260 |
| 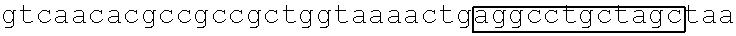 | 1302 |

Boxes: amino-acids introduced by genetic construction

### Amino-Acid Sequence:

[SEQ ID NO:10]

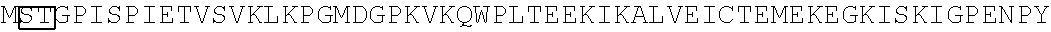

NTPVFAIKKKDSTKWRKLVDFRELNKRTQDFWEVQLGIPHPAGLKKKKSVTVLDVG
DAYFSVPLDEDFRKYTAFTIPSINNETPGIRYQYNVLPQGWKGSPAIFQSSMTKILEPF
RKQNPDIVIYQYMDDLYVGSDLEIGQHRTKIEELRQHLLRWGLTTPDKKHQKEPPFL

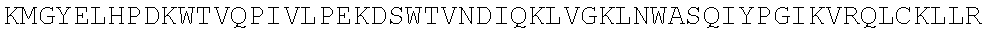

GTKALTEVIPLTEEAELELAENREILKEPVHGVYYDPSKDLIAEIQKQGQGQWTYQIY
QEPFKNLKTGKYARMRGAHTNDVKQLTEAVQKITTESIVIWGKTPKFKLPIQKETWE

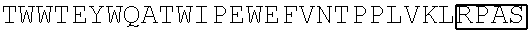

Boxes: amino-acids introduced by genetic construction.
K (Lysine): instead of Tryptophan (W). Mutation introduced to remove enzyme activity.

Alternatively, an F4 fusion which has been altered to remove a frameshift sequence so as to increase expression can be used. Such fusions are provided as SEQ ID NOs:11-12 for the nucleotide and protein sequences, respectively. The expressed protein retains all the immunogenicity of the F4co protein but is expressed more easily due to the frameshift deletion.

As discussed above, other F4 fusions which can be also be used include those derived from HIV sequences from different clades. For example, SEQ ID NOs:13-16 provide the nucleotide and protein sequences for F4 fusions based on HIV clade C. SEQ ID NOs:13-14 provide the nucleotide and amino acid sequence, respectively, of a clade C F4 sequence containing an option histidine tag (6×His) at the carboxy terminus for ease of purification. SEQ ID NOs:15-16 provide the nucleotide and amino acid sequences, respectively, of a clade C F4 sequence with a codon usage optimised for human expression. The amino acid sequences of SEQ ID NOs:14 and 16 are identical except that SEQ ID NO:14 contains the optional 6×His tag.

### 2.4 Preparation of F4co GMP Lots

F4co candidate vaccine was prepared and purified as previously described in U.S. Pat. No. 7,612,173 and U.S. Appl. No. 61/181,130. Briefly, a nucleic acid comprising the nucleotide sequence of SEQ ID NO:7 was cloned into a pET plasmid vector for expression in E. coli BLR(DE3) cells, which allows expression of the F4co polypeptide under control of a T7 promoter when induced by IPTG. After validation of correct expression, the cultures were scaled up into larger batches for the production of GMP batches. Three lots were purified and tested for yield, purity, and consistency, and met or exceeded standards for each characteristic.

### 3. Immunogenicity of F4 in Human Subjects

### 3.1 Methodology

### 3.1.1 Patient Population

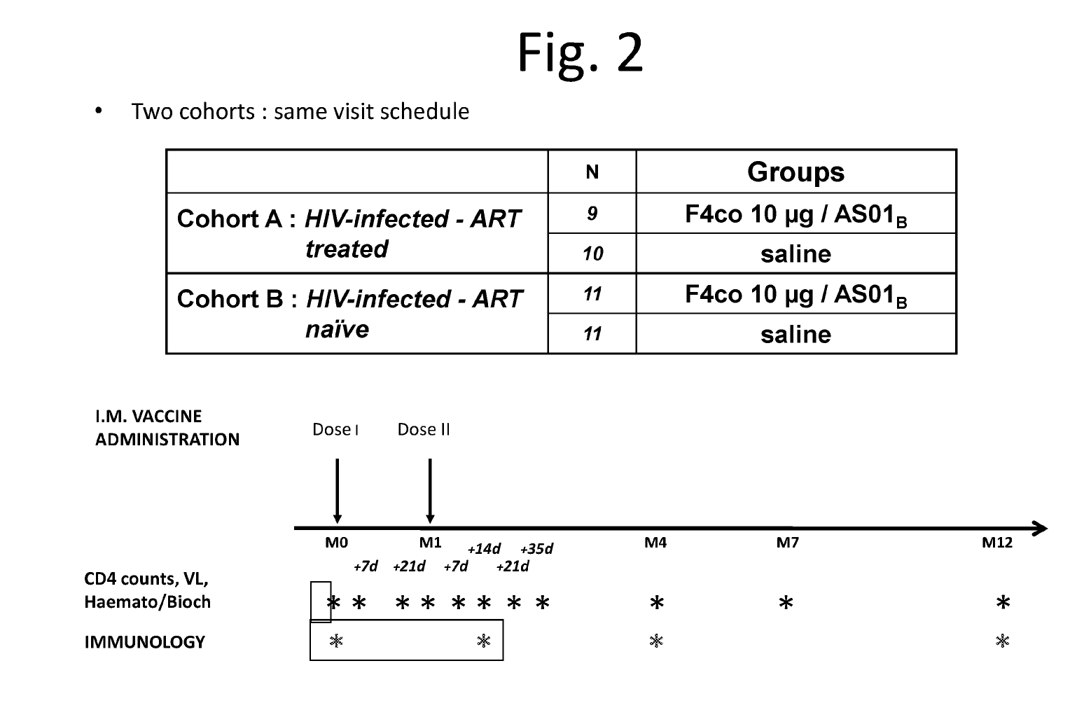

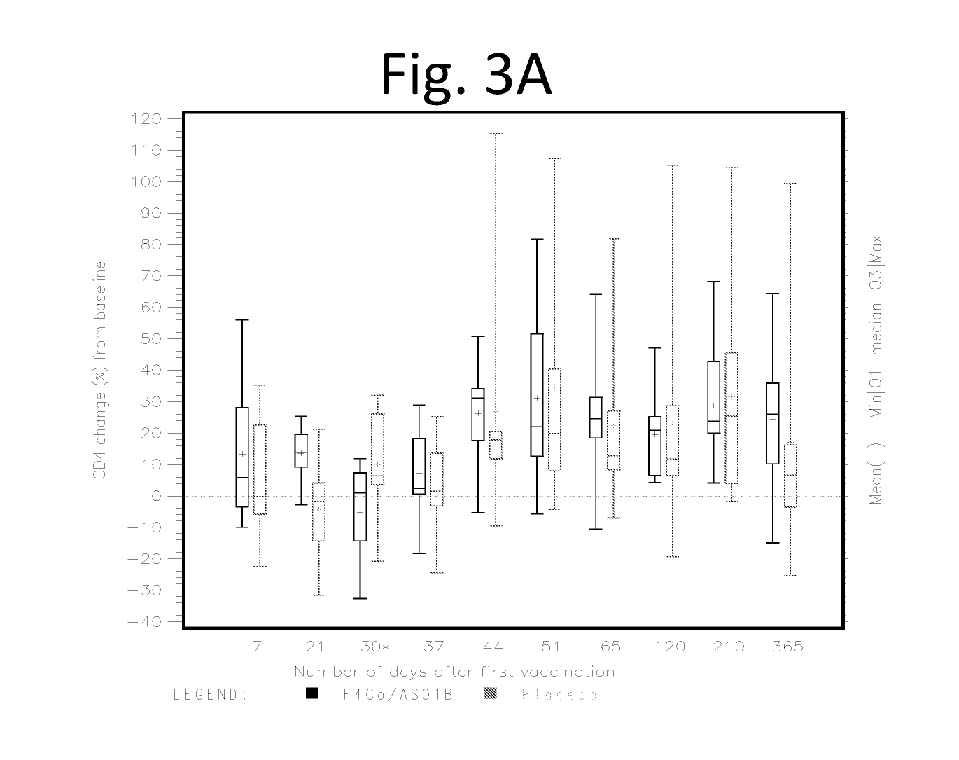

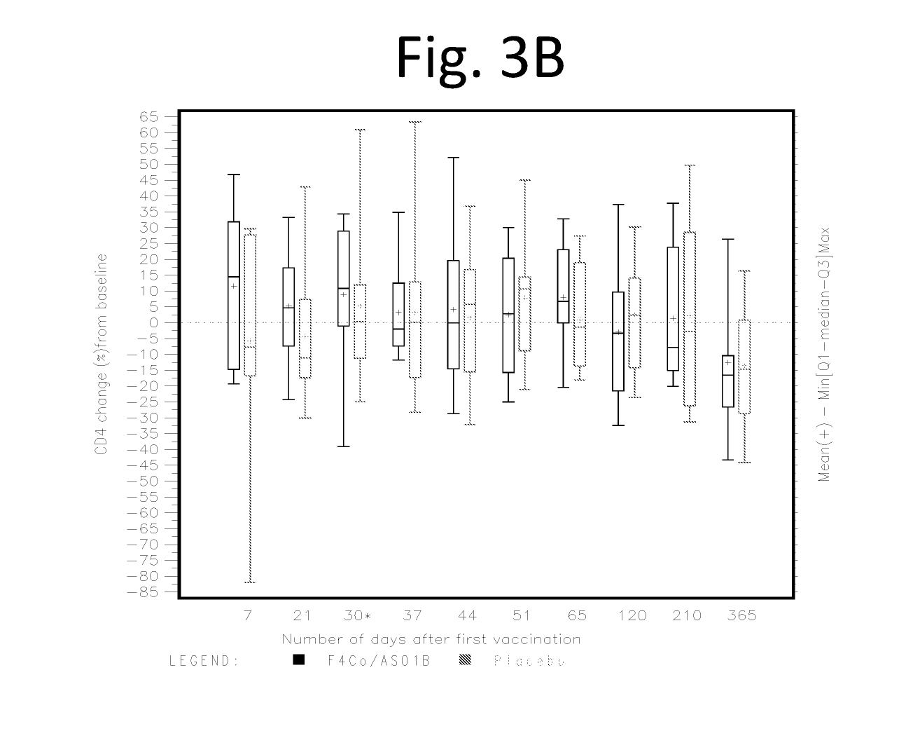

A placebo-controlled phase I, randomized observer-blind study of the immune response of HIV-infected subjects after vaccination with the F4co/ASO1_{B} vaccine candidate was performed at 6 centers in Germany (NCT00814762). The study was approved by the local independent ethics committee and the national regulatory authority, conducted in accordance with the Declaration of Helsinki and Good Clinical Practice guidelines and all subjects provided written informed consent. The primary study objective was to evaluate the reactogenicity and safety of the vaccine. Secondary objectives included assessment of HIV-1-specific CD4⁺ and CD8⁺ T-cell responses, CD4⁺ T-cell count and HIV viral load. Forty one subjects were enrolled, 19 ART-experienced and 22 ART-naïve (FIG. 2).

The demographic profile of the group which received the F4co/AS01_{B} vaccine was comparable to the group which received placebo for both ART-experienced and ART-naïve cohorts. The mean age in the two cohorts was 43.8 years and 37.6 years, respectively. All but one subject in each cohort were male. The cohorts were predominantly of White-Caucasian European heritage (94.7% and 90.9% of the subjects, respectively).

The mean time from HIV diagnosis to first vaccination was, respectively, 10.47 and 3.23 years. The CD4 cell nadir was approximately 250 cells/mm³ in the ART-experienced group and approximately 550 cells/mm³ in the ART-naïve group. The CD4 cell count at the time of the first vaccination ranged from 349 to 1055 cells/mm³ in the ART-experienced cohort and from 377 to 1188 cells/mm³ in ART-naïve subjects; in the latter cohort, the viral load at this time ranged from 2,280 to 69,400 copies/mL. All subjects were negative for HBsAg and HCV DNA. Additional information regarding the HIV status of the subjects can be found in Table 1.

TABLE 1
Summary of HIV history at baseline (total vaccinated cohort)

| | | ART-experienced | | | | ART-naïve | | | |
|---|---|---|---|---|---|---|---|---|---|
| | | F4co/AS01_{B} | | Placebo | | F4co/AS01_{B} | | Placebo | |
| | | N = 9 | | N = 10 | | N = 11 | | N = 11 | |
| | Parameters or | Value | | Value | | Value | | Value | |
| Characteristics | Categories | or n | % | or n | % | or n | % | or n | % |
| Duration between HIV | Mean | 13.11 | — | 8.10 | — | 3.55 | — | 2.91 | — |
| diagnosis | SD | 4.54 | — | 5.34 | — | 3.21 | — | 2.70 | — |
| and Dose 1 (years) | Median | 11.0 | — | 7.0 | — | 2.0 | — | 3.0 | — |
| | Minimum | 8.0 | — | 2.0 | — | 0.0 | — | 0.0 | — |
| | Maximum | 22.0 | — | 21.0 | — | 10.0 | — | 8.0 | — |
| CD4 count at baseline | Mean | 595.22 | — | 616.11 | — | 665.27 | — | 586.73 | — |
| | SD | 124.63 | — | 228.90 | — | 242.13 | — | 139.83 | — |
| | Median | 580.0 | — | 510.0 | — | 572.0 | — | 571.0 | — |
| | Minimum | 482 | — | 349 | — | 430 | — | 377 | — |
| | Maximum | 879 | — | 1055 | — | 1188 | — | 791 | — |
| HIV-1 viral load at baseline | Geometric | * | — | * | — | 20909.21 | — | 13649.02 | — |
| | mean | | | | | | | | |
| | Median | * | — | * | — | 21100.0 | — | 2280.0 | — |
| | Minimum | * | — | * | — | 6060 | — | 13200 | — |
| | Maximum | * | — | * | — | 69400 | — | 63900 | — |
| CD4 count nadir | Mean | 251.33 | — | 242.00 | — | 569.73 | — | 539.82 | — |
| | SD | 70.35 | — | 104.77 | — | 170.98 | — | 62.43 | — |
| | Median | 249.0 | — | 209.5 | — | 601.0 | — | 563.0 | — |
| | Minimum | 142 | — | 103. | — | 345 | — | 440 | — |
| | Maximum | 391 | — | 472. | — | 873 | — | 613 | — |
| Duration between nadir and | Mean | 7.89 | — | 5.30 | — | 1.36 | — | 0.36 | — |
| Dose 1 | SD | 3.52 | — | 3.37 | — | 1.91 | — | 0.67 | — |
| (years) | Median | 8.00 | — | 5.50 | — | 1.00 | — | 0.00 | — |
| | Minimum | 2.00 | — | 1.00 | — | 0.00 | — | 0.00 | — |
| | Maximum | 12.00 | — | 10.00 | — | 5.00 | — | 2.00 | — |
| HIV clade | B | 1 | 11.1 | 2 | 20.0 | 7 | 63.6 | 3 | 27.3 |
| | not known | 8 | 88.9 | 8 | 80.0 | 4 | 36.4 | 8 | 72.7 |
| Known HIV resistant | Yes | 2 | 22.2 | 3 | 30.0 | 1 | 9.1 | 0 | 0.0 |
| mutation | | | | | | | | | |

### 3.1.2 Formulation

The F4co vaccine candidate contained 10 μg per dose of F4 recombinant protein as active ingredient, adjuvanted with AS01_{B}. The vaccine antigen was prepared as a lyophilized pellet containing the F4 antigen in sucrose, EDTA, arginine, polysorbate 80 and sodium sulfite in phosphate buffer. The AS01_{B} liposome-based adjuvant system contains 50 μg 3-D-MPL and 50 μg QS21 and was prepared in accordance with Example 1 above.

The freeze-dried fraction containing the F4 antigen and the liquid fraction containing the AS01_{B} adjuvant system presented in a single-dose 3 ml glass vial were reconstituted by the person administering the vaccine shortly before injection. After dissolution of the vial contents, 0.5 ml of the reconstituted vaccine solution was withdrawn into a syringe for intramuscular administration. Alternatively, the same volume of saline was administered as a placebo in a similar manner.

### 3.1.3 Administration

The F4 vaccine candidate was administered twice with a 4 week interval between doses (FIG. 2), and subjects were monitored for safety. Reactogenicity was acceptable and did not increase upon repeat administration. Markers for HIV disease (CD4 counts and viral load) did not indicate aggravation of infection by vaccination. No serious adverse events related to vaccination were reported.

### 3.1.4 Safety

Local (injection site pain, redness, swelling) and general (fever, fatigue, headache, sweating, myalgia, gastrointestinal symptoms) solicited adverse events (AEs) were recorded for 7 days after each dose. Unsolicited AEs were recorded for 30 days after each vaccination. Severity of AEs was assessed using the National Institute of Allergy and Infectious Diseases Division of AIDS (DADS) AE grading system (NIAID 2004). Serious adverse events (SAEs) and the following predefined HIV-related AEs were assessed throughout the study period: 25% reduction in CD4⁺ cell count from baseline; a detectable viral load (50 copies/ml HIV RNA measured using an utrasensitive detection method) postvaccination in ART-experienced subjects/≧0.5 log increase in viral load postvaccination in ART-naïve subjects; change or initiation of ART; and abnormal biochemistry and/or hematology parameters (defined as on the DADS scale). Safety data were regularly reviewed by an independent data monitoring committee.

### 3.1.5 Viral Load

HIV viral load was tested with the Roche COBAS AMPLICOR™ HIV-1 Monitor Test v1.5 for the ART-experienced cohort and with the Roche COBAS AMPLIPREP™/COBAS TAQMAN™ HIV-1 Test v1.0 for the ART-naïve cohort.

### 3.1.6 CD4 Counts

CD4 counts were performed by using the commercial BD Multitest™ IMK kit (a four-colour assay) (Beckton Dickinson) and read on a BD FACS Calibur machine. During the course of the study, the method was upgraded by using the BDMultitest™ 6-color TBNK reagent and the BD FACS Canto II system after an extensive validation process.

### 3.1.7 Immune Response

HIV-specific CD4⁺ and CD8⁺ T-cell responses were evaluated by intracellular cytokine staining (ICS) following in vitro stimulation with p17, p24, RT and Nef peptide pools to assess the expression of interleukin-2 (IL-2), interferon-γ (IFN-γ), tumor necrosis factor-α (TNF-α) and CD40-ligand (CD40L) using peripheral blood mononuclear cells (PBMCs) isolated from venous blood as previously described (Van Braeckel et al. 2011). Results were expressed as the frequency of CD40L⁺CD4⁺ T-cells expressing at least IL-2, the cytokine co-expression profile and the percentage of responders after in vitro stimulation to each individual antigen and to at least 1, 2, 3 and all 4 antigens. If no cytokine secretion was detectable prevaccination, a subject was considered a responder if the proportion of CD40L⁺CD4⁺ T-cells expressing at least IL-2 was greater than or equal to the cut-off value of 0.03%. If cytokine secretion was detectable prevaccination, a subject was considered a responder if the proportion of CD40L⁺CD4⁺ T-cells expressing at least IL-2 was at least two-fold higher than baseline.

HIV-specific CD8+ T cell response was expressed as the frequency of CD8+ T cells expressing at least 1 cytokine amongst IL-2, TNF-α and IFN-γ. Additional exploratory analyses were performed in the ART-experienced cohort. PBMCs were stimulated in vitro either with peptide pools spanning the F4 antigen or with a selection of 6 immunodominant 9-mers peptides in HLA A*02 restricted population (RT₃₃₋₄₁, RT₁₂₇₋₁₃₅, RT₁₇₉₋₁₈₇, RT₃₀₉₋₃₁₇, p17₇₇₋₈₅, p24₁₉₋₂₇) (Frahm et al. 2008). Following the same procedure described above, cells were then stained with either a first panel of anti-CD8, CD3, 4-1BB, MIP-1β, IL-2, IFNγ antibodies and a pool of 6 tetramers (specific to the 6 immunodominant peptides) or with a second panel of anti-CD3, CD8, 4-1BB, IFNγ, perforin and granzyme B antibodies and the pool of 6 tetramers. Ex-vivo staining was also performed to analyze PD-1 expression, as well as activation markers such as CD38, HLA DR, CCR5 and Ki-67 on the total CD8⁺ T-cells or tetramer⁺ CD8⁺ T-cells.

### 3.1.8 Statistical Analysis

Analysis of safety and reactogenicity was performed on the total vaccinated cohort (i.e., all subjects who received at least one vaccine dose). The number and percentage of subjects reporting AEs were calculated with exact 95% confidence intervals (CI). Change in CD4 count and viral load from baseline were summarized for each treatment group in each cohort at each available time-point. For viral load change, reverse cumulative distribution curves were also derived.

Analysis of immunogenicity was performed on the according to protocol cohort (i.e., subjects who received both vaccine doses and complied with all study procedures for whom blood samples were available). Results were summarized within each group at each time-point using descriptive statistics for continuous variables and percentages (with 95% CI) for categorical variables. The F4-specific CD4+ T-cell response was estimated from the sum of the specific CD4+ T-cell frequencies in response to each individual antigen.

Exploratory comparisons between groups were derived for viral load, CD4+ cell count and cell-mediated immune (CMI) response, based on analysis of covariate (ANCOVA) models with the baseline as covariate for all time points, except baseline where no adjustment was performed (ANOVA), and using the arithmetic scale for the CD4+ cell count and the log scale for the viral load and CMI magnitude. No adjustments were made for multiplicity.

### 3.2 Results

### 3.2.1 CD4 Counts

CD4+ T cell counts and viral load levels were monitored and compared between treatment groups. CD4+ T cell count changes from baseline (study Day 0) are presented graphically in FIG. 3. In the ART-experienced cohort shown in panel A, the baseline CD4+ T cell count values were similar (mean values observed in F4co/AS01_{B} and placebo groups were, respectively, 595 and 616) and no differences were observed between the two treatment groups. An increase in CD4+ T cell count of ˜30-35% was observed in both groups after the second dose; the reason for this increase remains unclear. In ART-experienced subjects, this difference between the vaccine and the placebo groups remained significant up to month 4 (p<0.05), and F4-specific CD4⁺ T-cell responses were still detected in vaccine recipients at month 12. In the ART-naïve cohort (FIG. 3B), the baseline CD4+ T cell count values were similar (mean values observed in F4co/AS01_{B} and placebo groups were, respectively, 665 and 587); no consistent differences were observed between F4co/AS01_{B} and placebo groups over the study period. No association was observed between protective HLA-I alleles (HLA B*27, B*5801) or unfavourable HLA-I alleles (HLA B*35 types), haplotypes and randomization (placebo vs. vaccine), change in viral load or change in CD4⁺ T cell counts (data not shown). There were no patients carrying the protective HLA-I alleles B*57 and B*5802 in the study.

### 3.2.2 Viral Load

Except for two minor blips in the vaccine group and one minor blip in the placebo group, viral load remained suppressed in both groups of ART-experienced subjects over the 12 months of follow-up.

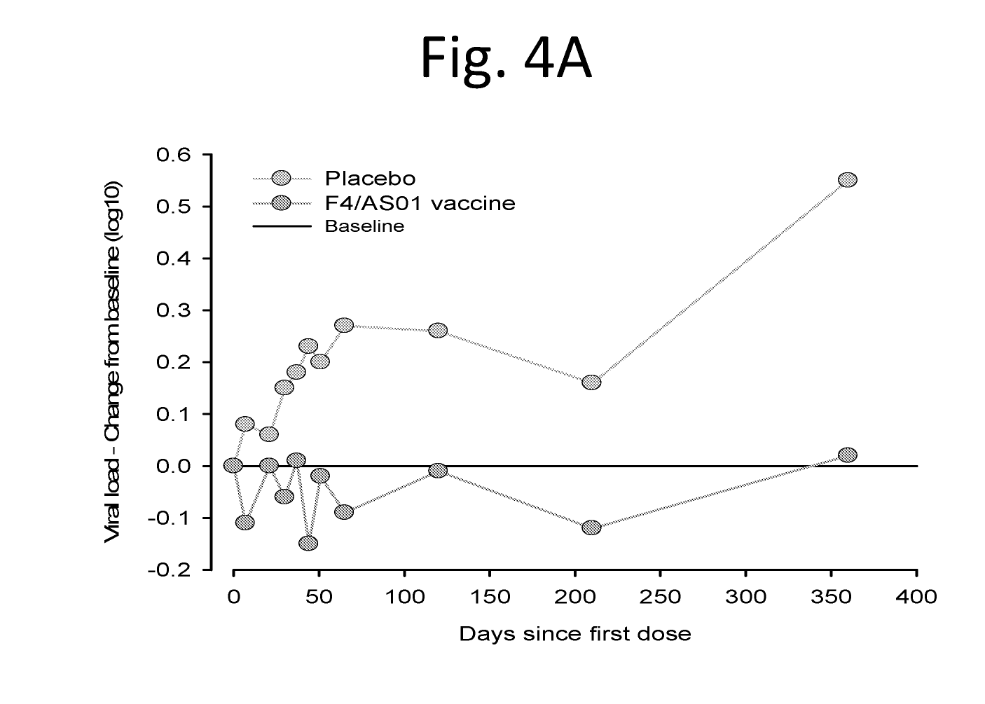

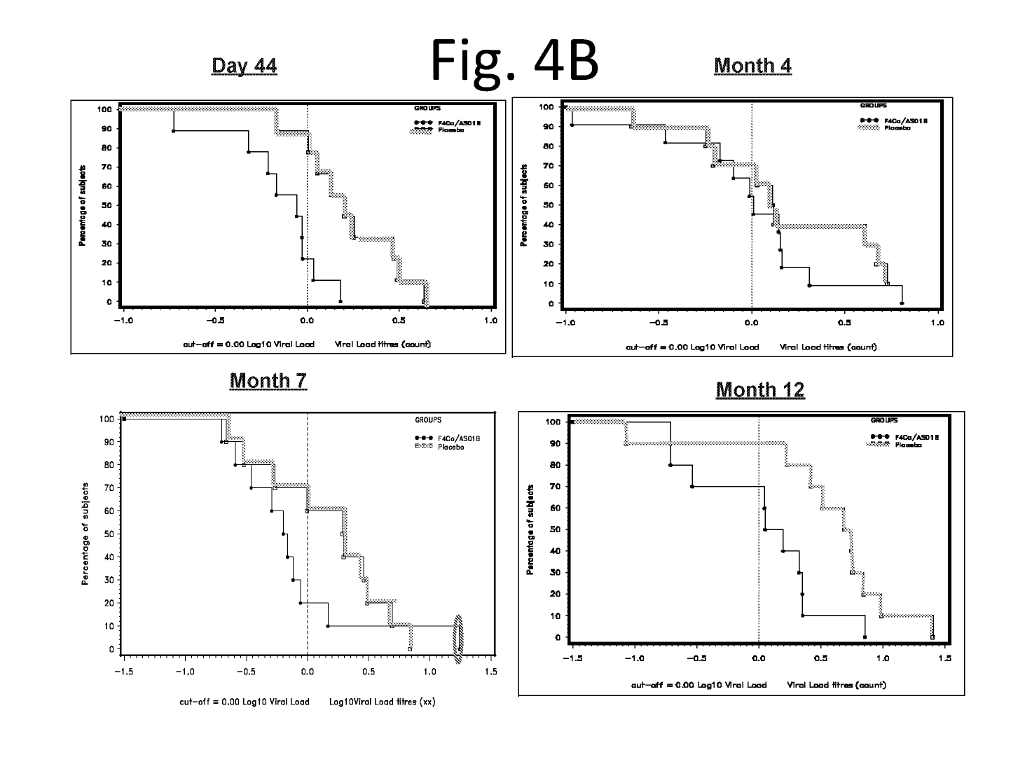

In ART-naïve subjects, a negative correlation was observed between the change in viral load from baseline and the frequency of CD4⁺ T-cells expressing at least IL-2 at two weeks post-dose 2 (FIG. 4A). Furthermore, ad hoc comparisons of change in mean viral load from baseline indicated a significant difference in favor of the vaccine group two weeks post-dose 2 (p<0.05), resulting in a shift between the groups in the reverse cumulative distribution curves (RCCs) for viral load change (FIG. 4B). This difference was sustained over the 12 months of follow-up, but was only statistically significant at two weeks post-dose 2.

### 3.2.3 Immunogenicity

### 3.2.3.1 T Cell Responses

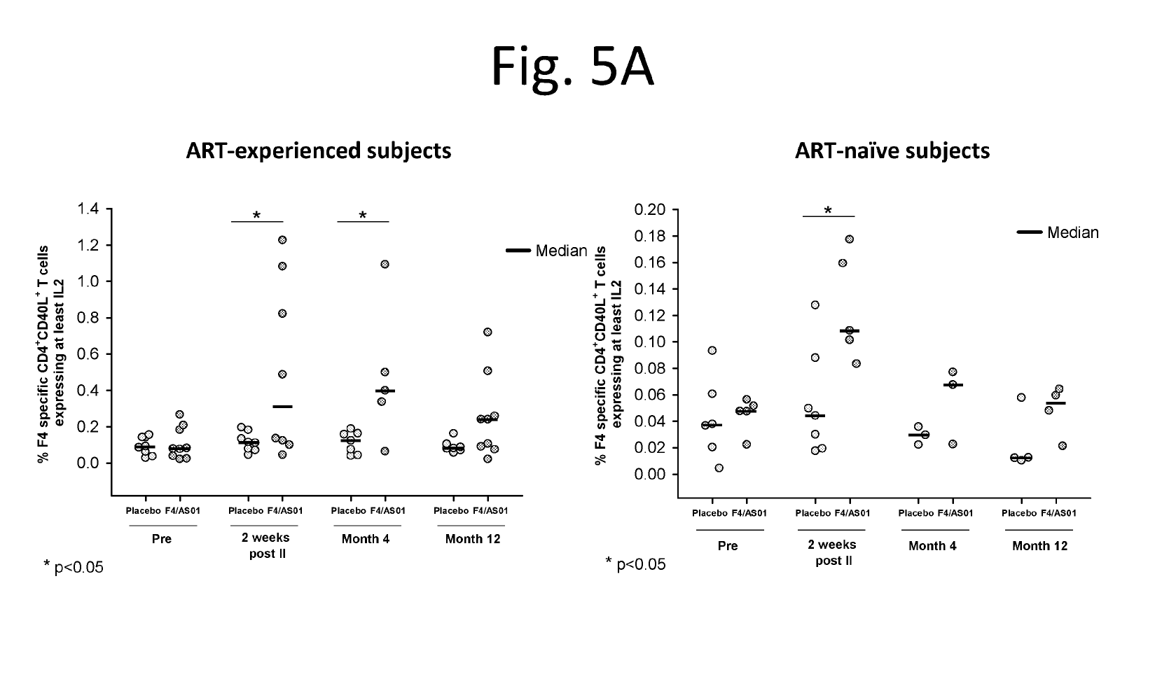

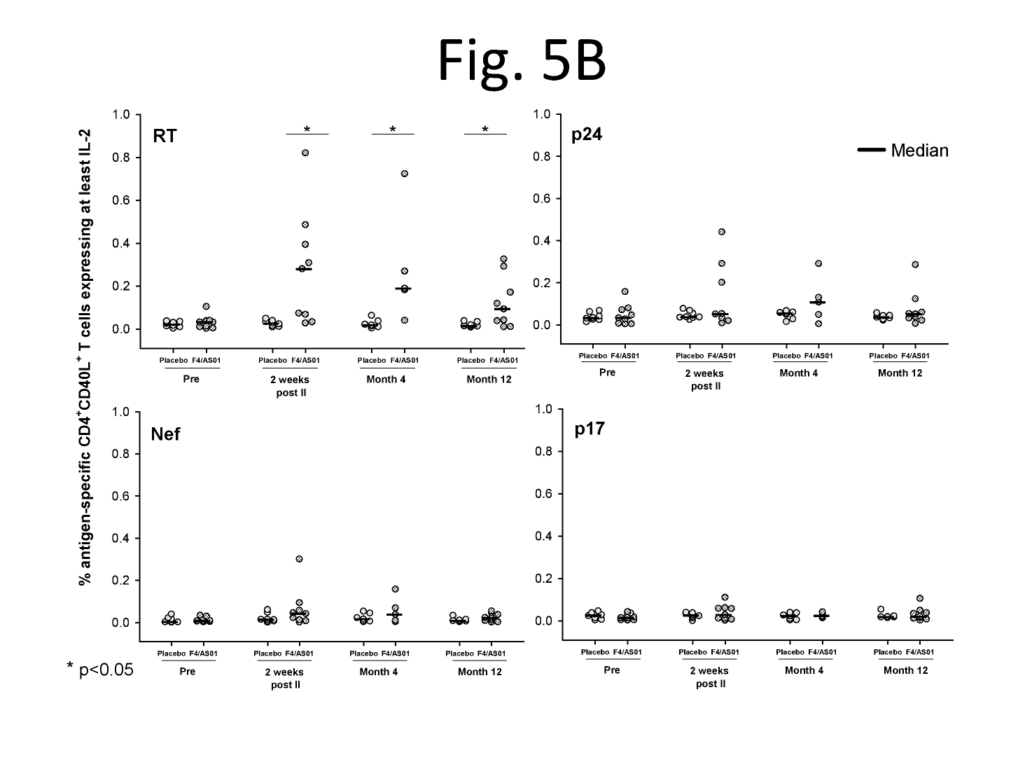

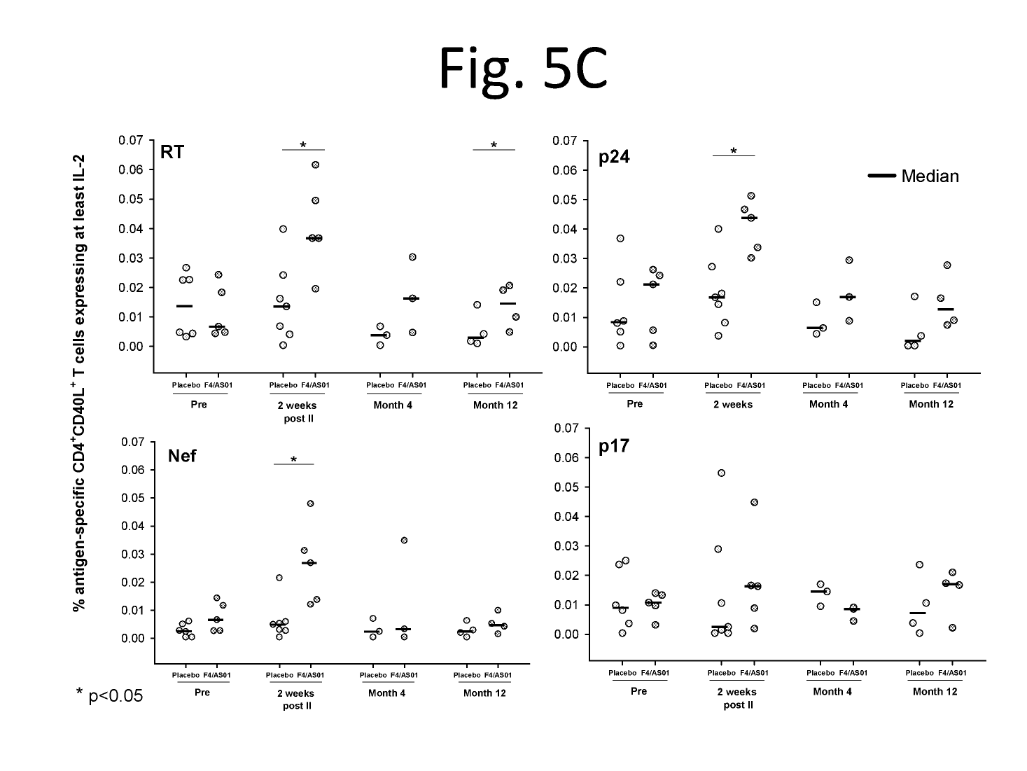

The frequency of F4-specific CD4+CD40L+ T-cells expressing at least IL-2 was significantly higher (p<0.05) in the vaccine group than in the placebo group two weeks post-dose 2 in both cohorts (FIG. 5A). Almost all vaccinees developed a CD4+ T-cell response two weeks post-dose 2 to at least one of the four antigens, with highest response rates against the RT and p24 antigens. In ART-experienced subjects, this difference between the vaccine and the placebo groups remained significant up to month 4 (p<0.05), and F4-specific CD4+ T-cell responses were still detected in vaccine recipients at month 12 (FIG. 5B). No significant differences were seen between the vaccine and the placebo groups at any later time-point in ART-naïve subjects (FIG. 5C).

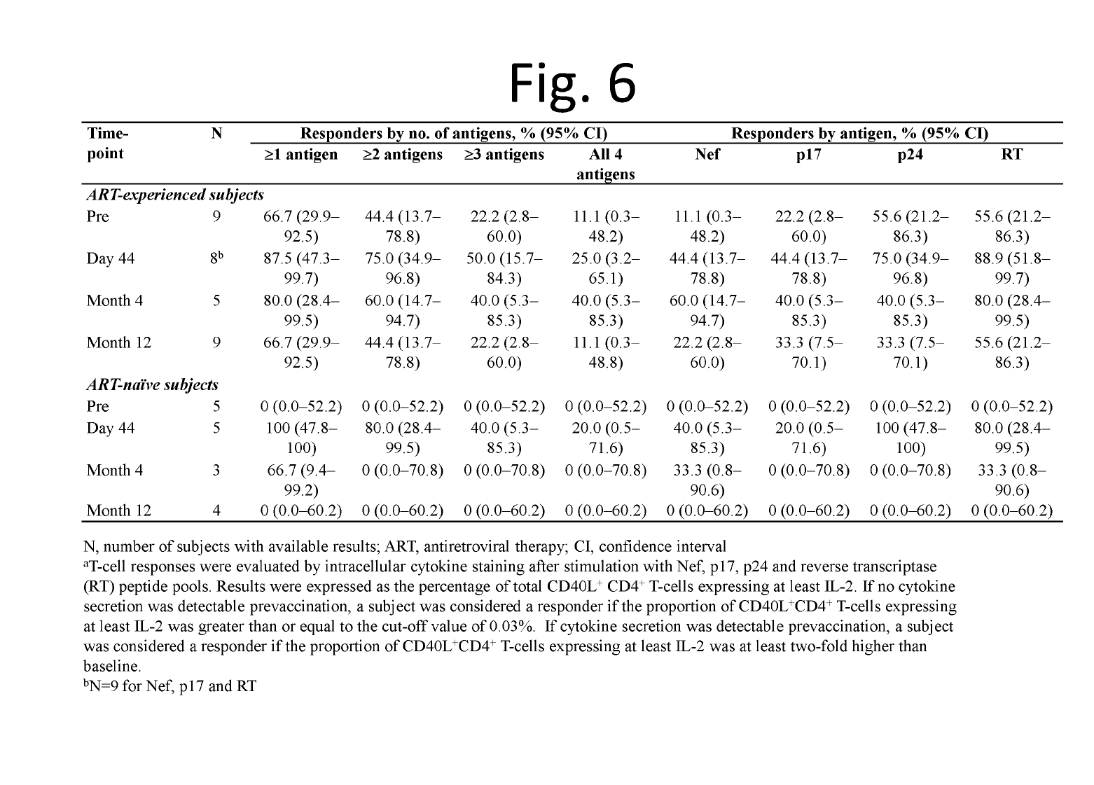

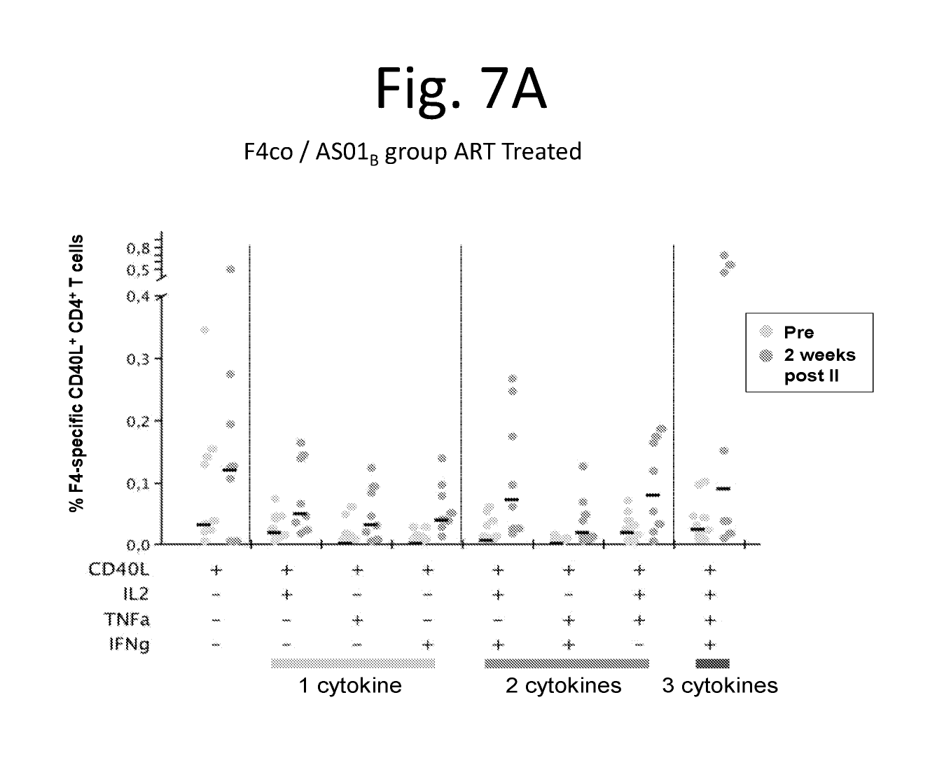

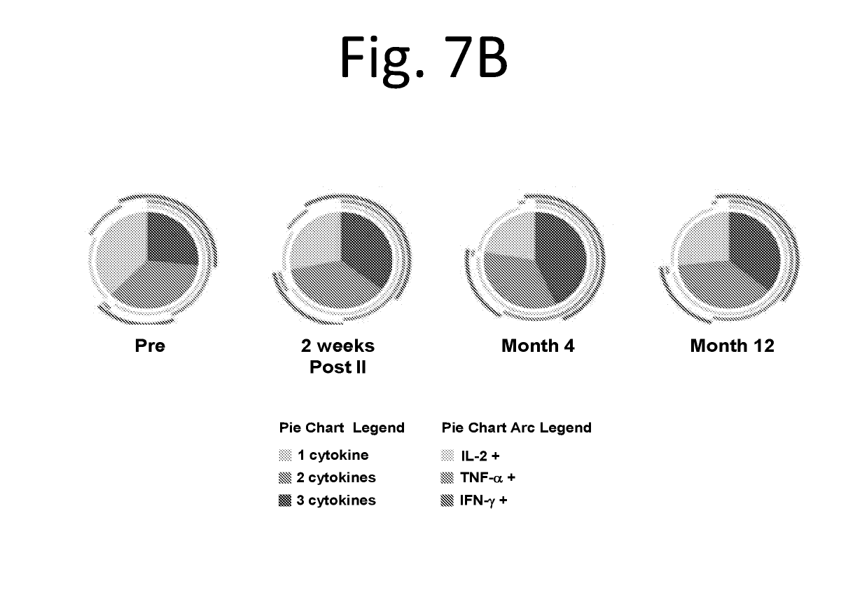

Pre-existing F4-specific CD4+CD40L+ T-cells expressing at least IL-2 were detected at a low frequency in both groups in ART-experienced and ART-naïve subjects prior to vaccination. Vaccine-induced CD4+ T-cells exhibited a polyfunctional phenotype (FIG. 6). In ART-experienced subjects, approximately 75% of F4-specific CD40L+CD4+ T-cells secreted at least 2 cytokines and approximately 35% secreted at least 3 cytokines and this cytokine coexpression profile was maintained until month 12. In ART-naïve subjects, approximately 50% of F4-specific CD40L+CD4+ T-cells secreted at least 2 cytokines and approximately 10% secreted at least 3 cytokines (FIGS. 7A-B).

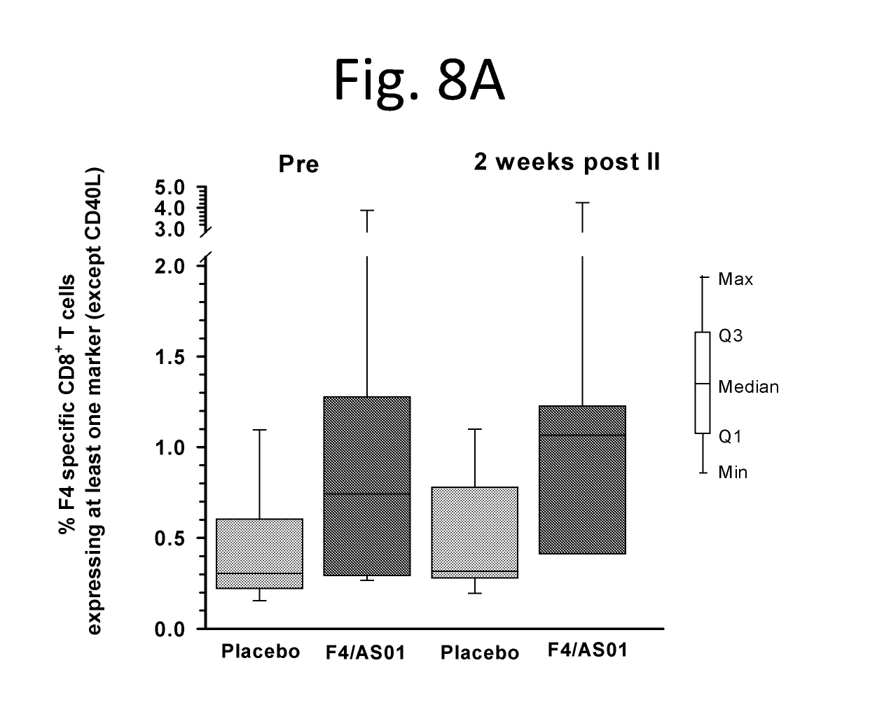

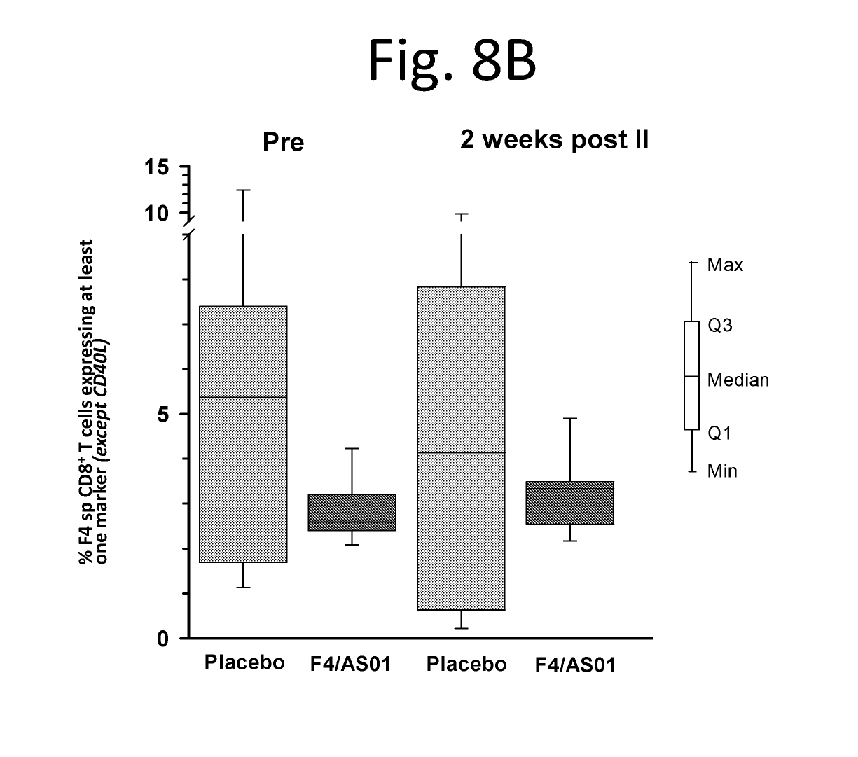

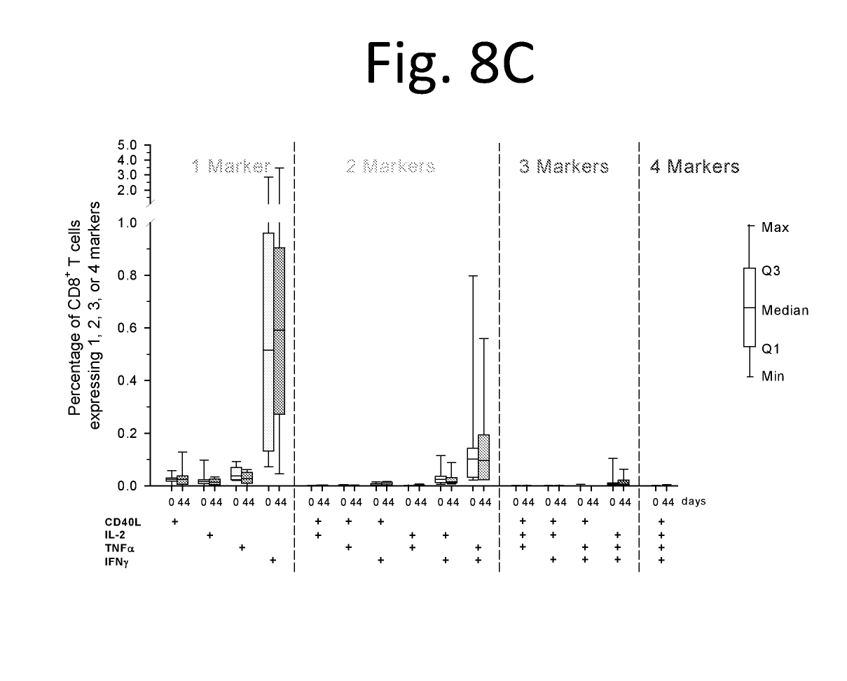

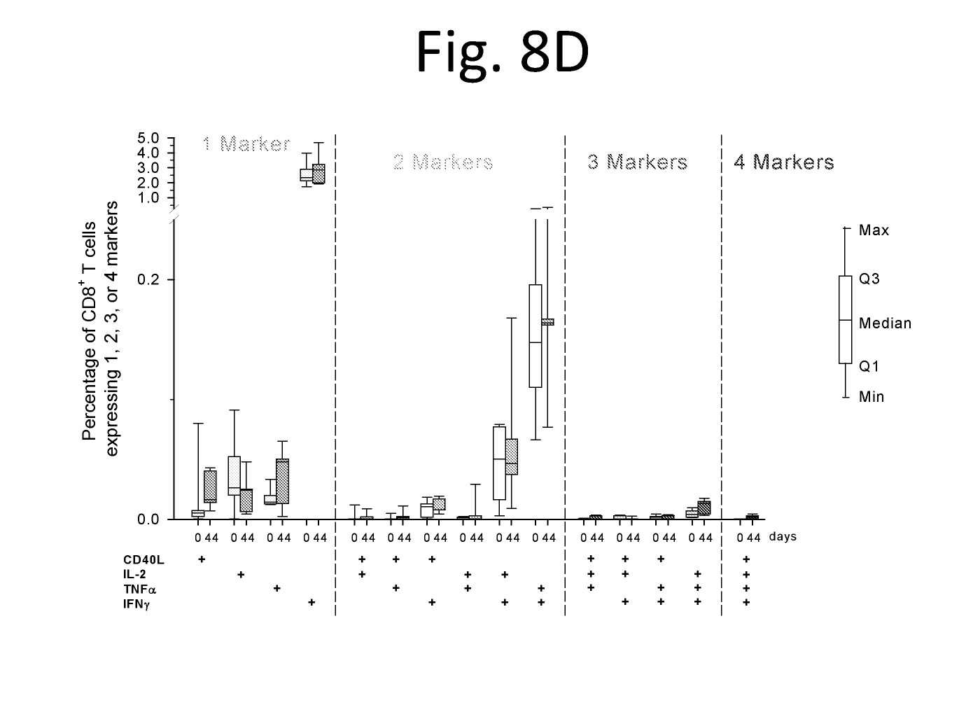

As shown in FIG. 8, high frequencies of CD8+ T-cells were detected pre-vaccination; these cells mainly expressed IFNγ. Administration of the F4co/AS01_{B} vaccine did not appear to have an effect on the median frequency, irrespective of the marker tested or stimulatory peptide pool used.

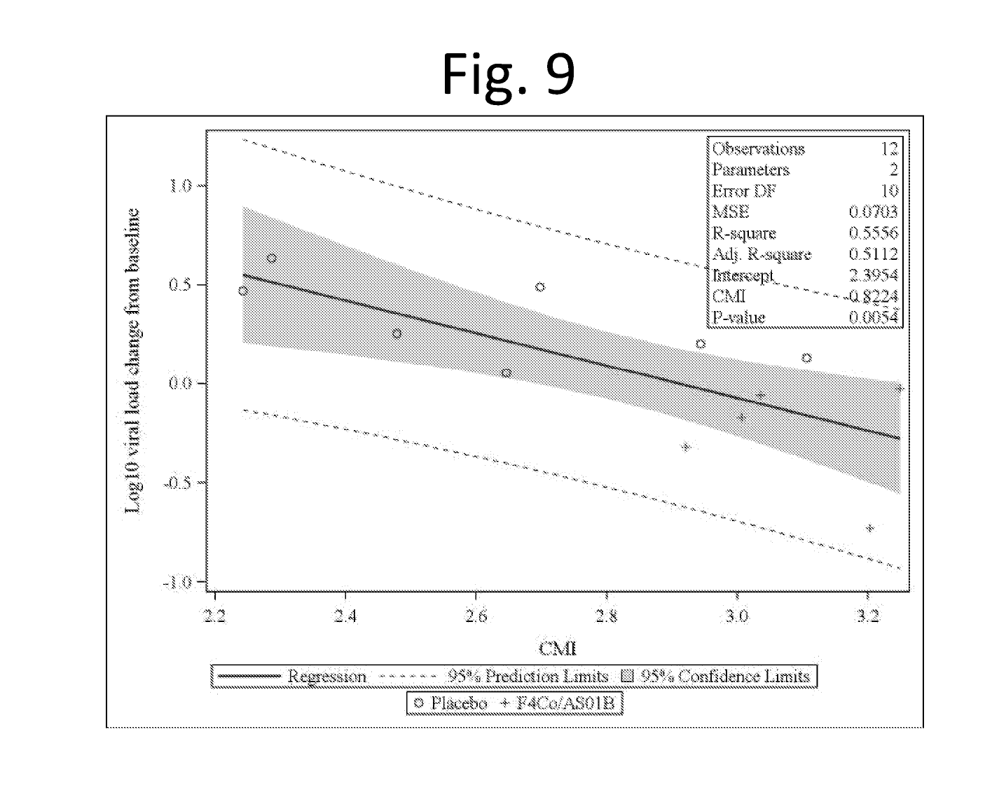

FIG. 9 shows the correlation between F4co-specific CD4+ T cells expressing at least IL-2 and viral load.

### 3.2.3.2 Antibody Responses

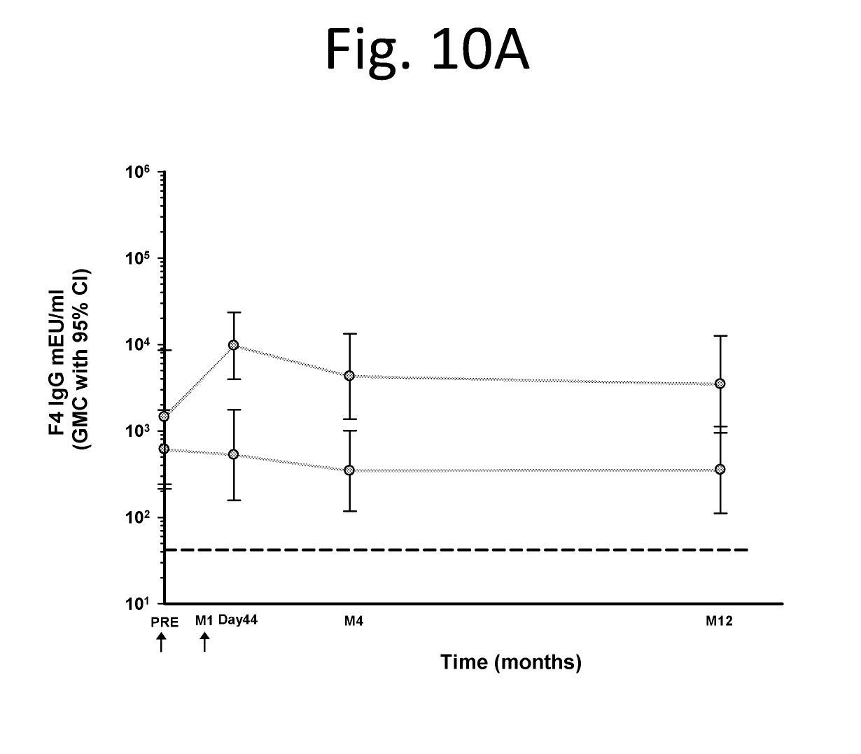

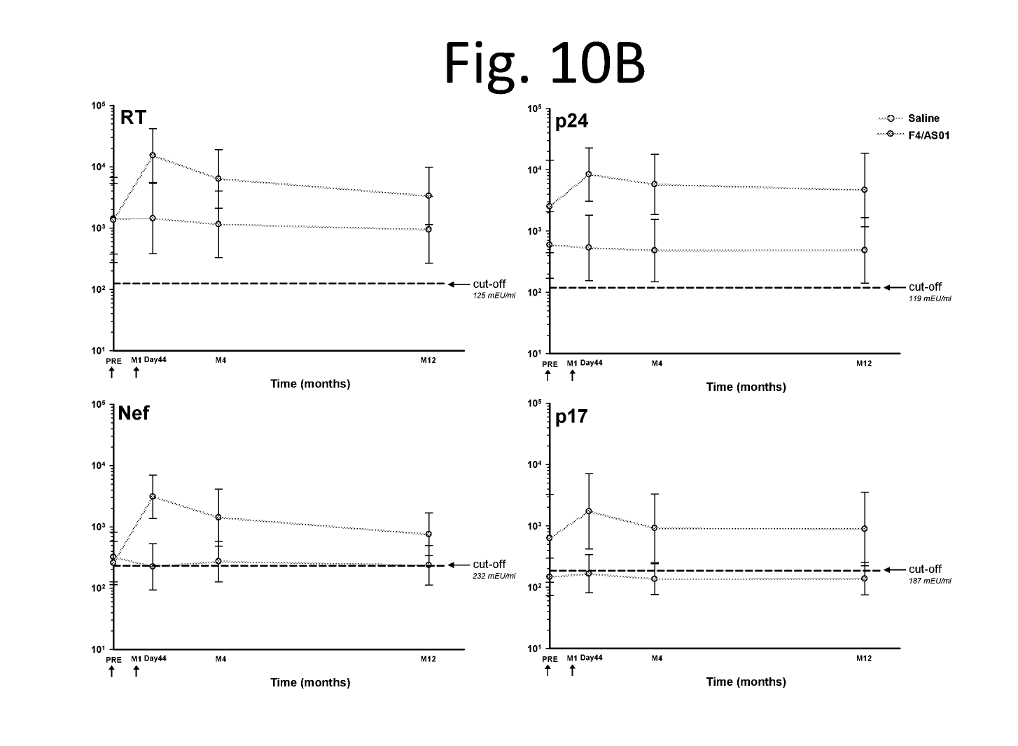

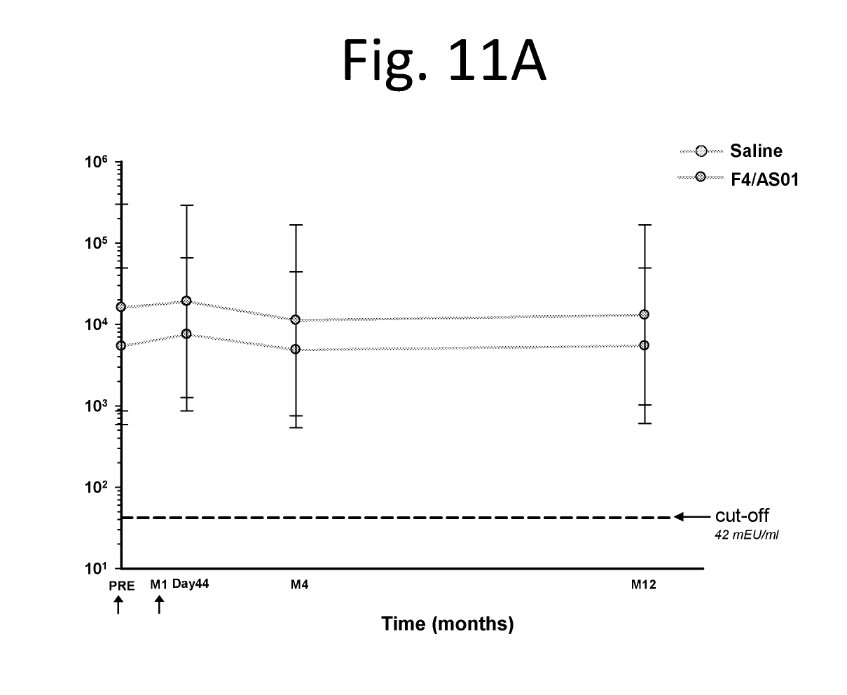

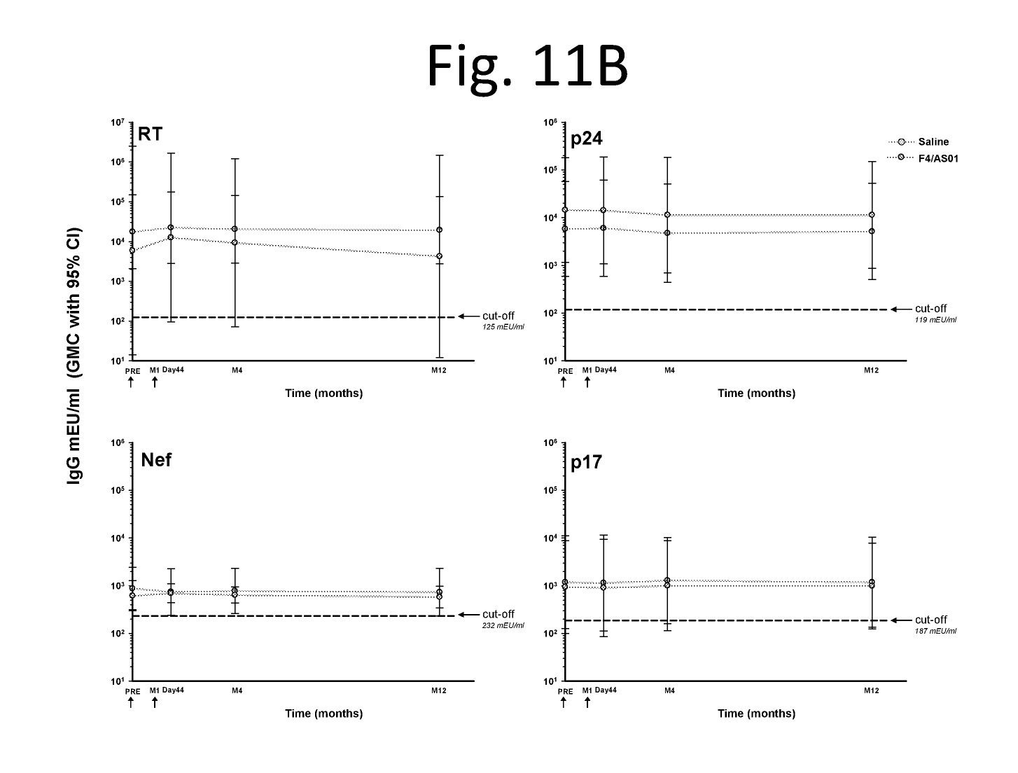

Both the ART-experienced and ART-naïve cohorts had high levels of circulating antibodies prior to vaccination. Antibody levels increased transiently following administration of the F4co/AS01_{B} vaccine in ART-experienced persons (FIGS. 10A-B). Increases were not observed in ART naïve subjects, who had higher pre-existing antibody levels (FIGS. 11A-B).

### 3.2.3.3 Summary of Immune Response

In the present study, two doses of the F4co/AS01_{B} vaccine were immunogenic in both ART-experienced and ART-naïve subjects. High CD4⁺ T-cell frequencies were observed with responses elicited against all vaccine antigens. By day 44, the overall immune response was statistically significantly higher in the F4co/AS01_{B} vaccine groups than in the placebo groups. The vaccine was more immunogenic in the ART-experienced cohort as compared to the ART-naïve cohort. The majority of antigen-specific CD40L⁺ CD4⁺ T-cells exhibited a polyfunctional phenotype, characterized by the combined expression of IL-2 with TNF-α and/or IFN-γ. A persistent trend of lower viral load as compared to placebo was observed in the ART-naïve cohort, while the ART-experienced cohort continued to have low viral loads.

### REFERENCES

Boaz M J, Waters A, Murad S, Easterbrook P J, Vyakarnam A. Presence of HIV-1 gag-specific IFN□+IL-2+ and CD28+IL-2+ CD4 T-cell responses is associated with nonprogression in HIV-1 infection. J Immunol. 2002; 169:6376-6385.
Centers for Disease Control and Prevention (CDC). Revised classification system for HIV infection and expanded surveillance case definition for AIDS among adolescents and adults. CDC, New York and Los Angeles; 1993. MMWR; 41 (no RR-17).
Dalsgaard et al. “Saponin adjuvants”, Archiv. für die gesamte Virusforschung, Vol. 44, Springer Verlag, Berlin, p243-254 (1974).
Frahm N, et al. Identification and optimal definition of HIV-derived cytotoxic T-lymphocyte (CTL) epitopes for the study of CTL escape, functional avidity and viral evolution. In: HIV Molecular Immunology 2008. Korber B T M, Brander C, Haynes B F, Koup R, Moore J P, Walker B D, and Watkins D I, editors. Los Alamos National Laboratory, Theoretical Biology and Biophysics, Los Alamos, N. Mex., USA; pp: 3-24.
Fraser et al. Variation in HIV-1 set-point viral load: epidemiological analysis and an evolutionary hypothesis. Proc Natl Acad Sci USA. 2007; 104(44):17441-6.
Harari A, Petitpierre S, Vallelian F, Skewed representation of functionally distinct populations of virus-specific CD4 T-cells in HIV infected subjects with progressive disease: changes after antiretroviral therapy, Blood. 2004; 103:966-72.
Iyasaere C, Tilton J C, Johnson A J, Diminished proliferation of human immunodeficiency virus-specific CD4+ T-cells is associated with diminished interleukin-2 (IL-2) production and is recovered by exogenous IL-2. J Virol. 2003; 77:10900-9.
Kannanganat S, Kapogiannis B G, Ibegbu C, et al. Human immunodeficiency virus type 1 controllers but not controllers maintain CD4 cells coexpressing three cytokines. J Virol. 2007; 81:12071-12076.
Kaufmann G R, Cunningham P, Zaunders J, et al. Impact of early HIV-1 RNA and T-lymphocyte dynamics during primary infection on the subsequent course of HIV-1 RNA levels and CD4+ T-lymphocytes counts in the first year of HIV-1 infection. JAIDS. 1999; 22:437-444.
Letvin N L. Animal models for the study of human immunodeficiency virus infections. Curr Opin Immunol. 1992; 4:481-485.
Lichterfeld M, Kaufmann D E, Yu X G, et al. Loss of HIV-1-specific CD8 T Cell Proliferation after acute HIV-1 Infection and Restoration by Vaccine-induced HIV-1-specific CD4 T Cells. Exp Med. 2004; 200(6):701-712.
Mellors J W et al. Prognosis in HIV-1 infection predicted by the quantity of virus in plasma. Science. 1996 May 24; 272(5265):1167-70.
Mosmann, T. R. and Coffman, R. L. TH1 and TH2 cells: different patterns of lymphokine secretion lead to different functional properties. Annual Review of Immunology (1989); 7: p145-173.
Pantaleo et al, N Engl J Med. (1993) 328(5):327-35.
Potter S J, Lacabaratz C, Lambotte O, et al. Preserved Central Memory and Activated Effector memory CD4+ T-cell subsets in human immunodeficiency virus controllers: an ANRS EP36 Study. J Virol. 2007; 89(24):13904-13915.
Thoelen et al. Safety and immunogenicity of a hepatitis B vaccine formulated with a novel adjuvant system. Vaccine (1998); 16:708-714.
Weiss R. How does HIV cause AIDS?. Science. 1993; 260:1673-1679.
World Health Organization (WHO). WHO Diseases Staging System for HIV Infection and Disease in Adults and Adolescents. WHO, Geneva, Switzerland, 2005.
Younes S A, HIV-1 viremia prevents the establishment of interleukin-2 producing HIV-specific memory CD4+ T-cells endowed with proliferative capacity. J Exp Med. 2003; 198:1909-22.
Van Braeckel E, Bourguignon P, Koutsoukos M, et al. An adjuvanted polyprotein HIV-1 vaccine induces polyfunctional cross-reactive CD4+ T cell responses in seronegative volunteers. Clin Infect Dis 2011; 52:522-31.
Voller et al. (eds). New Trends and Developments in Vaccines, University Park Press, Baltimore, Md., U.S.A. 1978
   Embodiments of the invention are further described in the following numbered paragraphs:
   Paragraph 1. A pharmaceutical composition comprising
   a. two or more HIV-1 antigens selected from the group consisting of Nef, Gag, and Pol;
   b. an adjuvant that induces a Th1 immune response; and
   c. a pharmaceutically acceptable excipient,
      for use in the maintenance of the viral load of an HIV-1 infected subject for at least four months after administration.
      Paragraph 2. A pharmaceutical composition comprising
   a. two or more HIV-1 antigens selected from the group consisting of Nef, Gag, and Pol;
   b. an adjuvant that induces a Th1 immune response; and
   c. a pharmaceutically acceptable excipient,
      for use in the reduction or maintenance of the viral load of an HIV-1 infected subject at or below 100,000 copies/ml for at least four months after administration.
      Paragraph 3. A pharmaceutical composition comprising
   a. two or more HIV-1 antigens selected from the group consisting of Nef, Gag, and Pol;
   b. an adjuvant that induces a Th1 immune response; and
   c. a pharmaceutically acceptable excipient,
      for use in enhancing the T cell response of an HIV-1 infected subject.
      Paragraph 4. The pharmaceutical composition of paragraph 3, wherein the enhanced T cell response is a higher percentage of either CD4+ T cells or CD8+ T cells from the subject that show specific recognition of at least one polypeptide of the pharmaceutical composition as compared to before administration of the pharmaceutical composition.
      Paragraph 5. The pharmaceutical composition of either of paragraphs 3 or 4, wherein the enhanced T cell response is a higher percentage of CD4+ T cells from the subject that express at least one, two or three activation markers as compared to before administration of the pharmaceutical composition.
      Paragraph 6. The pharmaceutical composition of any of paragraphs 1-5, wherein the subject is not on anti-retroviral therapy (ART).
      Paragraph 7. The pharmaceutical composition of any of paragraphs 1-6, wherein the HIV-1 infected subject is ART naïve.
      Paragraph 8. The pharmaceutical composition of any of paragraphs 1-6, wherein the HIV-1 infected subject discontinues ART prior to administration of the pharmaceutical composition.
      Paragraph 9. The pharmaceutical composition of any of paragraphs 1-6, wherein the HIV-1 infected subject is concurrently on ART.
      Paragraph 10. The pharmaceutical composition of any of paragraphs 1-9, wherein the viral load is maintained at or below 50,000 copies/ml, below 10,000 copies/ml, below 5000 copies/ml, below 1000 copies/ml, or below 500 copies/ml.
      Paragraph 11. The pharmaceutical composition of any of paragraphs 1-10, wherein the viral load is reduced after administration.
      Paragraph 12. The pharmaceutical composition of any of paragraphs 1-11, wherein the viral load is maintained or reduced for at least six months, at least twelve months, at least eighteen months, at least two years, at least three years, at least four years, at least five years, at least six years, at least seven years, at least eight years, at least nine years, or at least ten years.
      Paragraph 13. The pharmaceutical composition of any of paragraphs 1-12, wherein the pharmaceutical composition comprises Nef, Gag and Pol.
      Paragraph 14. The pharmaceutical composition of any of paragraphs 1-13, wherein Gag is p17, p24 or both.
      Paragraph 15. The pharmaceutical composition of any of paragraphs 1-14, wherein Pol is RT.
      Paragraph 16. The pharmaceutical composition of any of paragraphs 1-15, wherein the pharmaceutical composition comprises SEQ ID NO:8.
      Paragraph 17. The pharmaceutical composition of any of paragraphs 1-16, wherein the pharmaceutical composition comprises SEQ ID NO:10, SEQ ID NO:12, SEQ ID NO:14 and/or SEQ ID NO:16.
      Paragraph 18. The pharmaceutical composition of any of paragraphs 1-17, wherein the pharmaceutical composition further comprises Env.
      Paragraph 19. The pharmaceutical composition of any of paragraphs 1-18, wherein the adjuvant is one or more components selected from: an immunologically active saponin fraction, a lipopolysaccharide, an immunostimulatory oligonucleotide, and a sterol.
      Paragraph 20. The pharmaceutical composition of any of paragraphs 1-19, wherein the adjuvant comprises an immunologically active saponin fraction and a lipopolysaccharide.
      Paragraph 21. The pharmaceutical composition of any of paragraphs 1-20, wherein the adjuvant comprises QS21 and/or a lipid A derivative.
      Paragraph 22. The pharmaceutical composition of paragraph 21, wherein the lipid A derivative is 3D-MPL.
      Paragraph 23. The pharmaceutical composition of any of paragraphs 1-22, wherein the adjuvant comprises CpG.
      Paragraph 24. The pharmaceutical composition of any of paragraphs 19-23, wherein the sterol is cholesterol.
      Paragraph 25. The pharmaceutical composition of any of paragraphs 1-24, wherein the adjuvant further comprises a liposome carrier.
      Paragraph 26. The pharmaceutical composition of any of paragraphs 1-25, wherein the pharmaceutical composition is administered once to the subject.
      Paragraph 27. The pharmaceutical composition of any of paragraphs 1-26, wherein the pharmaceutical composition is administered two or more times to the subject.
      Paragraph 28. The pharmaceutical composition of any of paragraphs 1-27, wherein the immunogenic composition is administered as either the priming dose or boosting dose of a prime-boost regimen.
      Paragraph 29. A method for stabilizing or inhibiting the increase in the viral load in a subject infected with HIV-1 comprising

1) selecting a subject infected with HIV-1; and

2) administering to the subject an immunogenic composition comprising
a. two or more HIV-1 antigens selected from the group consisting of Nef, Gag, and Pol;
b. an adjuvant that induces a Th1 immune response; and
c. a pharmaceutically acceptable excipient,
   wherein after administration the viral load of the subject remains stable or decreases for at least four months as compared to before administration.
   Paragraph 30. A method for the prevention of the onset of clinical HIV disease in a subject infected with HIV-1 comprising

1) selecting a subject infected with HIV-1; and

2) administering to the subject an immunogenic composition comprising
a. two or more HIV-1 antigens selected from the group consisting of Nef, Gag, and Pol;
b. an adjuvant that induces a Th1 immune response; and
c. a pharmaceutically acceptable excipient,
   wherein the viral load of the subject remains below 100,000 copies/ml for at least four months after administration.
   Paragraph 31. A method for the prevention of the progression of HIV disease in a HIV-1 infected subject comprising

1) selecting a subject infected with HIV-1; and

2) administering to the subject an immunogenic composition comprising
a. two or more HIV-1 antigens selected from the group consisting of Nef, Gag, and Pol;
b. an adjuvant that induces a Th1 immune response; and
c. a pharmaceutically acceptable excipient,
   wherein the viral load of the subject remains stable after administration of the immunogenic composition.
   Paragraph 32. A method for inducing an immune response in a subject infected with HIV-1 comprising

1) selecting a subject infected with HIV-1; and

2) administering to the subject an immunogenic composition comprising
a. two or more HIV-1 antigens selected from the group consisting of Nef, Gag, and Pol;
b. an adjuvant that induces a Th1 immune response; and
c. a pharmaceutically acceptable excipient,
   wherein after the administration of step 2), a higher percentage of CD4+ T cells from the subject show specific recognition of at least one polypeptide of the immunogenic composition is increased as compared to before the administration.
   Paragraph 33. A method for inducing an immune response in a subject infected with HIV-1 comprising

1) selecting a subject infected with HIV-1; and

2) administering to the subject an immunogenic composition comprising
a. two or more HIV-1 antigens selected from the group consisting of Nef, Gag, and Pol;
b. an adjuvant that induces a Th1 immune response; and
c. a pharmaceutically acceptable excipient,
   wherein after the administration of step 2), a higher percentage of CD4+ T cells from the subject express at least one, two or three activation markers selected from the group consisting of CD40L, IL-2, TNFα and IFNγ as compared to before administration.
   Paragraph 34. The method of any of paragraphs 29-33, wherein the subject is not on anti-retroviral therapy (ART).
   Paragraph 35. The method of any of paragraphs 29-34, wherein the HIV-1 infected subject is ART naïve.
   Paragraph 36. The method of any of paragraphs 29-34, wherein the HIV-1 infected subject discontinues ART prior to administration of the pharmaceutical composition.
   Paragraph 37. The method of any of paragraphs 29-34, wherein the HIV-1 infected subject is concurrently on ART.
   Paragraph 38. The method of any of paragraphs 29-37, wherein the viral load is maintained at or below 50,000 copies/ml, below 10,000 copies/ml, below 5000 copies/ml, below 1000 copies/ml, or below 500 copies/ml.
   Paragraph 39. The method of any of paragraphs 29-38, wherein the viral load is reduced after administration.
   Paragraph 40. The method of any of paragraphs 29-39, wherein the viral load is maintained or reduced for at least six months, at least twelve months, at least eighteen months, at least two years, at least three years, at least four years, at least five years, at least six years, at least seven years, at least eight years, at least nine years, or at least ten years.
   Paragraph 41. The method of any of paragraphs 29-40, wherein the pharmaceutical composition comprises Nef, Gag and Pol.
   Paragraph 42. The method of any of paragraphs 29-41, wherein Gag is p17, p24 or both.
   Paragraph 43. The method of any of paragraphs 29-42, wherein Pol is RT.
   Paragraph 44. The method of any of paragraphs 29-43, wherein the pharmaceutical composition comprises SEQ ID NO:8.
   Paragraph 45. The method of any of paragraphs 29-44, wherein the pharmaceutical composition comprises SEQ ID NO:10, SEQ ID NO:12, SEQ ID NO:14 and/or SEQ ID NO:16.
   Paragraph 46. The method of any of paragraphs 29-45, wherein the pharmaceutical composition further comprises Env.
   Paragraph 47. The method of any of paragraphs 29-46, wherein the adjuvant is one or more components selected from: an immunologically active saponin fraction, a lipopolysaccharide, an immunostimulatory oligonucleotide, and a sterol.
   Paragraph 48. The method of any of paragraphs 29-47, wherein the adjuvant comprises an immunologically active saponin fraction and a lipopolysaccharide.
   Paragraph 49. The method of any of paragraphs 29-48, wherein the adjuvant comprises QS21 and/or a lipid A derivative.
   Paragraph 50. The method of paragraph 49, wherein the lipid A derivative is 3D-MPL.
   Paragraph 51. The method of any of paragraphs 29-50, wherein the adjuvant comprises CpG.
   Paragraph 52. The method of any of paragraphs 47-51, wherein the sterol is cholesterol.
   Paragraph 53. The method of any of paragraphs 29-52, wherein the adjuvant further comprises a liposome carrier.
   Paragraph 54. The method of any of paragraphs 29-53, wherein the pharmaceutical composition is administered once to the subject.
   Paragraph 55. The method of any of paragraphs 29-54, wherein the pharmaceutical composition is administered two or more times to the subject.
   Paragraph 56. The method of any of paragraphs 29-55, wherein the immunogenic composition is administered as either the priming dose or boosting dose of a prime-boost regimen.

What is claimed is:

1-9. (canceled)

10. A method for stabilizing or inhibiting the increase in the viral load in a subject infected with HIV-1 comprising
1) selecting a subject infected with HIV-1; and
2) administering to the subject an immunogenic composition comprising
a. two or more HIV-1 antigens selected from the group consisting of Nef, Gag, and Pol;
b. an adjuvant that induces a Th1 immune response; and
c. a pharmaceutically acceptable excipient,
wherein after administration the viral load of the subject remains stable or decreases for at least four months as compared to before administration.

11. A method for the prevention of the onset of clinical HIV disease in a subject infected with HIV-1 comprising
1) selecting a subject infected with HIV-1; and
2) administering to the subject an immunogenic composition comprising
a. two or more HIV-1 antigens selected from the group consisting of Nef, Gag, and Pol;
b. an adjuvant that induces a Th1 immune response; and
c. a pharmaceutically acceptable excipient,
wherein the viral load of the subject remains below 100,000 copies/ml for at least four months after administration.

12. A method for the prevention of the progression of HIV disease in a HIV-1 infected subject comprising
1) selecting a subject infected with HIV-1; and
2) administering to the subject an immunogenic composition comprising
a. two or more HIV-1 antigens selected from the group consisting of Nef, Gag, and Pol;
b. an adjuvant that induces a Th1 immune response; and
c. a pharmaceutically acceptable excipient,
wherein the viral load of the subject remains stable or decreases after administration of the immunogenic composition.

13. A method for inducing an immune response in a subject infected with HIV-1 comprising
1) selecting a subject infected with HIV-1; and
2) administering to the subject an immunogenic composition comprising
a. two or more HIV-1 antigens selected from the group consisting of Nef, Gag, and Pol;
b. an adjuvant that induces a Th1 immune response; and
c. a pharmaceutically acceptable excipient.
wherein after the administration of step 2), a higher percentage of CD4+ T cells from the subject
i) show specific recognition of at least one polypeptide of the immunogenic composition is increased as compared to before the administration, or
ii) express at least one, two or three activation markers selected from the group consisting of CD40L, IL-2, TNFα and IFNγ as compared to before administration.

14. The method of claim 10, wherein:
a. the subject is not on anti-retroviral therapy (ART); and/or
b. the HIV-1 infected subject is
i) ART naïve,
ii) discontinues ART prior to administration of the pharmaceutical composition, or
iii) is concurrently on ART.

15-18. (canceled)

19. The method of claim 10, wherein the pharmaceutical composition comprises Nef, Gag and Pol.

20. The method of claim 19, wherein Gag is p17, p24 or both.

21. The method of claim 19, wherein Pol is RT.

22. The method of claim 19, wherein the pharmaceutical composition comprises SEQ ID NO:8.

23. The method of claim 19, wherein the pharmaceutical composition comprises SEQ ID NO:10, SEQ ID NO:12, SEQ ID NO:14 and/or SEQ ID NO:16.

24. The method of claim 10, wherein the pharmaceutical composition further comprises Env.

25. The method of claim 10, wherein the adjuvant is one or more components selected from: an immunologically active saponin fraction, a lipopolysaccharide, an immunostimulatory oligonucleotide, and a sterol.

26. The method of claim 25, wherein the adjuvant comprises QS21 and a lipid A derivative 3D-MPL.

27. The method of claim 25, wherein the adjuvant comprises CpG.

28. The method of claim 25, wherein the sterol is cholesterol.

29. The method of claim 25, wherein the adjuvant further comprises a liposome carrier.

30. The method of claim 10, wherein the pharmaceutical composition is administered once to the subject.

31. The method of claim 10, wherein the pharmaceutical composition is administered two or more times to the subject.

32. The method of claim 10, wherein the immunogenic composition is administered as either the priming dose or boosting dose of a prime-boost regimen.

33. The method of claim 11, wherein the viral load is maintained at or below 50,000 copies/ml, 10,000 copies/ml, 5000 copies/ml, 1000 copies/ml or 500 copies/ml.

34. The method of claim 10, wherein the viral load is stable or decreases for at least six months, at least twelve months, at least eighteen months, at least two years, at least three years, at least four years, at least five years, at least six years, at least seven years, at least eight years, at least nine years, or at least ten years.
